(19) United States
(12) Patent Application Publication
(10) Pub. No.: US 2026/0166217 A1
(43) Pub. Date: Jun. 18, 2026

(54) MEDICANT DELIVERY SYSTEM, METHODS OF PROVIDING RECOMMENDATIONS REGARDING MEDICANT DELIVERY, AND RELATED SYSTEMS AND METHODS

(71) Applicant: Insulet Corporation, Acton, MA (US)

(72) Inventors: Mert Sevil, Manchester, NH (US); Ashutosh Zade, San Diego, CA (US); Joon Bok Lee, Acton, MA (US); William J. Whiteley, Knoxville, TN (US)

(21) Appl. No.: 19/423,716

(22) Filed: Dec. 17, 2025

Related U.S. Application Data
(60) Provisional application No. 63/735,195, filed on Dec. 17, 2024.

(51) Int. Cl. A61M 5/142

(52) U.S. Cl. CPC A61M 5/142; A61M 2005/14208; A61M 2202/0007; A61M 2202/04; A61M 2205/3303; A61M 2205/502; A61M 2230/201

(57) ABSTRACT

A system for administration of medicament to a user-body may include an automated medicament delivery device configured to receive therapy data comprising data acquired over a previous at least seven-day period, analyze the therapy data to identify at least one behavior trend represented in the at least seven-day period, based at least partially on the identified at least one behavior trend, predict at least one event, and based at least partially on the predicted at least one event, generate a request to adjust operation of the automated medicament delivery device.

### CROSS-REFERENCE TO RELATED APPLICATIONS

This application claims the benefit under 35 U.S.C. § 119 (e) of U.S. Provisional Patent Application Ser. No. 63/735,195, filed Dec. 17, 2024, the disclosure of which is hereby incorporated herein in its entirety by this reference.

The present disclosure generally relates to medicament delivery systems. More particularly, the present disclosure relates to automated medicament delivery algorithms and personalizing the algorithms.

### BACKGROUND

Automated medicament delivery devices (“AMD,” e.g., an Automated Insulin Delivery (AID) device, without limitation) are often used to administer medicaments to the body of a patient via a cannula inserted into the body to treat medical conditions (e.g., Type 1 Diabetes, without limitation).

A bolus of medicament (e.g., a correction bolus or a carbohydrate bolus (also called a “meal bolus”), without limitation) may be delivered by the AMD to the user-body as an immediate bolus (i.e., a specified amount of medicament administered in a single dose), an extended bolus (a specified amount of medicament administered as a sequence of discrete doses at a constant rate over a set duration of time), or a combination bolus (a portion of a specified amount of medicament administered immediately in a single dose and the remainder administered over a set duration of time).

### BRIEF SUMMARY

Some embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive therapy data comprising data acquired over a previous at least seven-day period; analyze the therapy data to identify at least one behavior trend represented in the at least seven-day period; based at least partially on the identified at least one behavior trend, predict at least one event; and based at least partially on the predicted at least one event, generate a request to adjust operation of the automated medicament delivery device.

One or more embodiments include a method for managing medicant delivery to a user, the method comprising: receiving therapy data comprising data acquired over a previous at least seven-day period; analyzing the therapy data to identify at least one behavior trend represented in the at least seven-day period; based at least partially on the identified at least one behavior trend, predicting at least one event; and providing an indication of the predicted event.

Some embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive therapy data comprising at least one of insulin-on-board data or blood glucose data acquired over a previous at least seven-day period; analyze the therapy data to identify at least one behavior trend represented in the previous at least seven-day period; and based at least partially on the identified at least one behavior trend, generate a request to adjust operation of the automated medicament delivery device.

One or more embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive therapy data comprising data acquired over a previous at least seven-day period; associate each data point of the therapy data with a time cluster of a plurality of time clusters; for each time cluster of the plurality of time clusters, extract at least one therapy variable; determine at least one statistical feature of the at least one therapy variable; based at least partially on the determined at least one statistical feature, identify at least one behavior trend represented in the at least seven-day period; based at least partially on the identified at least one behavior trend, predict at least one event; and based at least partially on the predicted at least one event, generate a request to adjust operation of the automated medicament delivery device.

Some embodiments include a method for managing medicant delivery to a user, the method comprising: receiving therapy data comprising data acquired over a previous at least seven-day period; associating each data point of the therapy data with a time cluster of a plurality of time clusters; for each time cluster of the plurality of time clusters, extracting at least one therapy variable; determining at least one statistical feature of the at least one therapy variable; based at least partially on the determined at least one statistical feature, identifying at least one behavior trend represented in the at least seven-day period; based at least partially on the identified at least one behavior trend, predicting at least one event; and providing an indication of the predicted event.

One or more embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive therapy data comprising at least one of insulin-on-board data or blood glucose data acquired over a previous at least seven-day period; associate each data point of the therapy data with a time cluster of a plurality of time clusters; for each time cluster of the plurality of time clusters, extract at least one therapy variable; determine at least one statistical feature of the at least one therapy variable; based at least partially on the determined at least one statistical feature, identify at least one behavior trend represented in the at least seven-day period; and based at least partially on the identified at least one behavior trend, generate a request to adjust operation of the automated medicament delivery device.

Some embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive current therapy data comprising data acquired over a most recent 24-hr period; receive historical therapy data comprising data acquired over at least a seven-day period prior to the 24-hr period; estimate time-series coefficients for data points of the historical therapy data by fitting a time-series model to the data points of the historical therapy data; based at least partially on the determined time-series coefficients, predict at least one event; based at least partially on the current therapy data, assess a prediction error of the predicted at least one event; adjust the time-series model based at least partially on the assessed prediction error; based on the adjusted time-series model, predict at least one future event; and based at least partially on the predicted at least one future event, generate a request to adjust operation of the automated medicament delivery device.

One or more embodiments include a method for managing medicant delivery to a user, the method comprising: receiving historical therapy data comprising data acquired over at least a seven-day period prior to a current 24-hr period; fitting a time-series model to the data points of the historical therapy data; utilizing the fitted time-series model to predict at least one event; and providing an indication of the predicted event.

Some embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive historical therapy data comprising data acquired over at least a seven-day period; fit a time-series model to the data points of the historical therapy data; utilize the fitted time-series model to predict at least one event; and based at least partially on the predicted at least one event, generate a request to adjust operation of the automated medicament delivery device.

One or more embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive current therapy data comprising data acquired over a most recent 24-hr period; receive historical therapy data comprising data acquired over at least a seven-day period prior to the 24-hr period; fit at least a portion of the current therapy data to a first curve; fit at least one portion of the historical therapy data to at least one second curve; compare the first curve to the at least one second curve; based at least partially on the comparison of the first curve to the at least one second curve, predict at least one event; and based at least partially on the predicted at least one event, generate a request to adjust operation of the automated medicament delivery device.

Some embodiments include a method for managing medicant delivery to a user, the method comprising: receiving current therapy data comprising data acquired over a most recent 24-hr period; receiving historical therapy data comprising data acquired over at least a seven-day period prior to the 24-hr period; fitting at least a portion of the current therapy data to a first curve; fitting at least one portion of the historical therapy data to at least one second curve; comparing the first curve to the at least one second curve; based at least partially on the comparison of the first curve to the at least one second curve, predicting at least one event; and providing an indication of the predicted event.

One or more embodiments include a system for administration of medicament to a user-body, the system comprising: an analyte sensor; and an automated medicament delivery device comprising: at least one processor; and at least one non-transitory computer-readable storage medium storing instructions thereon that, when executed by the at least one processor, cause the automated medicament delivery device to: receive current therapy data comprising data acquired over a most recent 24-hr period; receive historical therapy data comprising data acquired over at least a seven-day period prior to the 24-hr period; fit at least a portion of the current therapy data to a first curve; fit each of a plurality of portions of the historical therapy data to a respective second curve of a plurality of second curves; compare the first curve to each of the plurality of second curves; identify a second curve of the plurality of second curves that exhibit a smallest total difference relative to the first curve based at least partially on the identified second curve, predict at least one event; and based at least partially on the predicted at least one event, generate a request to adjust operation of the automated medicament delivery device.

Other technical features may be readily apparent to one skilled in the art from the following figures, descriptions, and claims.

Within the scope of this application, it should be understood that the various aspects, embodiments, examples and alternatives set out herein, and individual features thereof may be taken independently or in any possible and compatible combination. Where features are described with reference to a single aspect or embodiment, it should be understood that such features are applicable to all aspects and embodiments unless otherwise stated or where such features are incompatible.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a schematic diagram illustrating a medicament delivery system in accordance with one or more examples;

FIG. 2 is a block diagram of a medicament delivery system for controlled administration of medicament in accordance with one or more examples;

FIG. 3 shows a flowchart of method according to one or more embodiments of the disclosure;

FIG. 4 shows a flowchart of method according to one or more embodiments of the disclosure;

FIG. 5 shows a flowchart of method according to one or more embodiments of the disclosure; and

FIG. 6 shows a flowchart of method according to one or more embodiments of the disclosure.

### DETAILED DESCRIPTION

While the specification concludes with claims particularly pointing out and distinctly claiming what are regarded as embodiments of the present disclosure, various features and advantages may be more readily ascertained from the following description of example embodiments when read in conjunction with the accompanying drawings, in which:

Illustrations presented herein are not meant to be actual views of any particular automated medicament delivery device, insulin pump, component, or system, but are merely idealized representations that are employed to describe embodiments of the disclosure. Additionally, elements common between figures may retain the same numerical designation for convenience and clarity.

The following description provides specific details of embodiments. However, a person of ordinary skill in the art will understand that the embodiments of the disclosure may be practiced without employing many such specific details. Indeed, the embodiments of the disclosure may be practiced in conjunction with conventional techniques employed in the industry. In addition, the description provided below does not include all the elements that form a complete structure or assembly. Only those process acts and structures necessary to understand the embodiments of the disclosure are described in detail below. Additional conventional acts and structures may be used. The drawings accompanying the application are for illustrative purposes only and are thus not drawn to scale.

As used herein, the terms “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of” and grammatical equivalents thereof.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “configured” refers to a size, shape, material composition, and arrangement of one or more of at least one structure and at least one apparatus facilitating operation of one or more of the structure and the apparatus in a predetermined way.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

As used herein, the term “and/or” includes any and all combinations of one or more of the associated listed items.

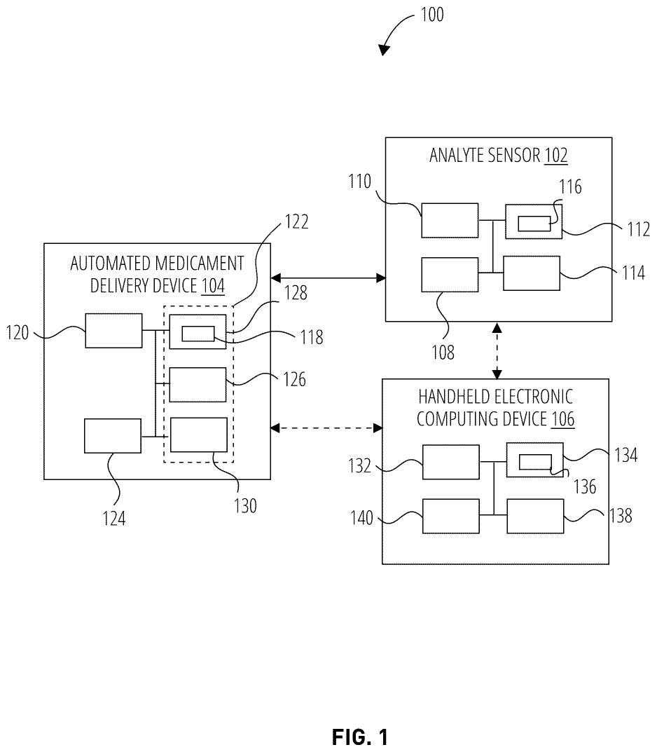

FIG. 1 is a schematic diagram showing a system 100 for administration of medicament to a user-body, in accordance with one or more examples.

In one or more examples, a system 100 may be capable of one or more modes of operation of administration of medicament (e.g., one or more distinct modes of operation, without limitation). Non-limiting examples of the one or more modes of operation include: fully automated administration of medicament, partially automated administration of medicament, and/or manual administration of medicament. In one or more examples, the system 100 may be capable of alternating between the multiple (e.g., two or more, without limitation) modes of operation. As a non-limiting example, the system 100 may alternate between one or more of: fully automated operation, partially automated operation, and/or manual operation.

The system 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, and combinations or sub combinations thereof, without limitation. Analyte values may include values representative of amounts of one or more analytes present within a user-body and values at least partially based on the same, such as, an A1C value, a blood glucose value (e.g., milligrams per decaliter (mg/dL), without limitation), an insulin-to-carbohydrate (I:C) ratio, or any combination thereof, without limitation.

The system 100 may also administer an amount of medicament at least partially based on user inputs (e.g., a user defined bolus amount or details related to a meal consumed or about to be consumed, such as number of carbohydrates, amount of fat, and amount of protein, without limitation). As used herein, administration of medicament responsive to a user input may be referred to as manual medicament delivery or manual delivery.

Non-limiting examples of medicaments administrable by system 100 include: insulin, glucagon-like peptide-1 receptor agonist (GLP-1), glucose-dependent insulinotropic polypeptide (GIP), or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated. system 100.

The system 100 includes an analyte sensor 102 and an automated medicament delivery device 104. The system 100 may optionally include a handheld electronic computing device 106.

The analyte sensor 102 may be configured to obtain data related to one or more analytes within the user-body (“analyte data”). In various examples, the analyte data may include one or more analyte values. In various examples, the analyte sensor 102 is an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEXCOM® G6 manufactured by Dexcom, without limitation).

The analyte sensor 102 may include a filament 108 and various electronic components. The filament 108 may be configured to obtain data related to one or more analytes within a user-body and provide the data to the various electronic components of the analyte sensor 102. The filament 108 may be configured to obtain the data directly from fluids of a user-body, including without limitation interstitial fluids of a user-body, from tissue of a user-body, combinations thereof, or in any other manner known in the art.

The analyte sensor 102 may include one or more processors 110, a memory 112, and communication equipment 114. The memory 112 may be coupled to the one or more processors 110. The memory 112 may be used for storing data, metadata, and programs for execution by the one or more processors 110. The memory 112 may include storage for storing data or instructions 116. The instructions 116 may include instructions for processing data obtained via the filament 108. When the instructions 116 are executed by the one or more processors 110, the instructions 116 cause the one or more processors 110 to process the data obtained via the filament 108. The instructions 118 may be implemented in hardware (e.g., one or more hardware processors of the one or more processors 110, such as an integrated circuit, application specific integrated circuit (ASIC), digital signal processor (DSP), or other logic circuit, without limitation), implemented in software (e.g., firmware, software, machine code, applications, without limitation), or a combination thereof. The instructions 116 for processing the data obtained via the filament 108 may include one or more instructions respectively for determining analyte values at least partially based on the data, or for sending the data, analyte values or both to the automated medicament delivery device 104 and/or the handheld electronic computing device 106.

The communication equipment 114 is configured to facilitate communication (e.g., a device or interface for wired communication, wireless communication, both wired and wireless communication, without limitation) of the analyte sensor 102 with other devices, including the automated medicament delivery device 104 and/or the handheld electronic computing device 106, without limitation. Such communication may be according to any appropriate wired or wireless communication protocol, such as WI-FI®, BLUETOOTH®, near-field communication (NFC), radio-frequency identification (RFID), or any other radio-frequency, infrared, or optical communication technology.

The automated medicament delivery device 104 may be configured to administer medicament to a user-body, such as subcutaneously into the user-body, without limitation, in accordance with one or more examples. In one or more examples, the automated medicament delivery device 104 may offer one or more modes of operation for administration of medicament to a user-body. When operating in some of the modes of operation, the automated medicament delivery device 104 may administer medicament at least partially responsive to analyte values, including without limitation analyte values received from analyte sensor 102. When operating in some further modes of operation, the automated medicament delivery device 104 may administer medicament at least partially responsive to user input. When operating some yet further modes of operation, the automated medicament delivery device 104 may administer medicament at least partially responsive to both analyte values and user input. Non-limiting examples of the one or more modes of operation offered by the automated medicament delivery device 104 include: fully automated administration of medicament, partially automated administration of medicament, or manual administration of medicament.

When operating in an operative mode that includes manual administration of medicament, the automated medicament delivery device 104 may administer medicament solely in response to a user input (e.g., delivers medicament in response to a user confirmation of delivery of medicament or in response to a user instruction to delivery medicament, without limitation). When operating in an operative mode that includes fully automated administration of medicament, the automated medicament delivery device 104 may administer medicament solely in response to analyte values (e.g., delivers medicament in response to one or more analyte values, without limitation). When operating in an operative mode that includes partially automated administration of medicament, the automated medicament delivery device 104 may administer medicament in response to analyte values and user input (e.g., delivers medicament in response to a user input and an analyte value, or alternately delivers medicament in response to a user input or in response to analyte values, without limitation).

Medicament administration may include administration of a basal amount of medicament regularly delivered a control interval (e.g., at a determined basal rate, without limitation) to keep analyte levels stale and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user defined bolus (e.g., responsive to user inputs provided, such as a user defined bolus amount or details related to a meal consumed or about to be consumed, such as number of carbohydrates, amount of fat, and amount of protein, without limitation).

The automated medicament delivery device 104 may include a delivery system 120, a controller 122 and a power source 124. The controller may include one or more processors 126, a memory 128, and communication equipment 130. In one or more examples, the automated medicament delivery device 104, or portions thereof, may include a wearable device and may be secured to a user-body (e.g., secured via one or more adhesive layers attaching the automated medicament delivery device 104 to the skin of the user-body or a material that is secured to the user body, without limitation).

In various examples, the delivery system 120 is configured to cause an amount of medicament to move (e.g., flow, without limitation) toward and/or into a user-body.

In various examples, delivery system 120 may deliver amounts of medicament at least partially responsive to requests. In various examples, instructions 118 of the memory 128 may include instructions for determining and generating requests for delivery system 120. In various examples, instructions 118 may include instructions for determining one or more amounts of medicament, determining a timing for delivery of one or more amounts of medicament, and for generating one or more requests for delivery system 120 related to the same. When such instructions of instructions 118 are executed by one or more processors 126, the one or more processors 126 determine the amounts of medicament and timing of delivery, generate requests for the delivery system 120 at least partially based on the determined amounts and timing, and provide the requests to delivery system 120. In some embodiments, the requests and/or instructions for generating requests may be received from the handheld electronic computing device 106. Furthermore, activity (e.g., determined and generated requests for delivery system 120, administered doses, etc.) of the automated medicament delivery device 104 may be stored in the memory 128.

The communication equipment 130 is configured to facilitate communication (e.g., wireless communication, without limitation) of the automated medicament delivery device 104 with other devices, including without limitation communication between analyte sensor 102 and the automated medicament delivery device 104 and communication between the automated medicament delivery device 104 and the handheld electronic computing device 106. The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In various examples, the communication equipment 130 includes an Internet of Things (IOT) Subscriber Identity Module (SIM) card (e.g., a machine-to-machine SIM card, a Universal Integrated Circuit Card, without limitation).

The power source 124 is configured to supply power to the delivery system 120 and the various electronic components, such as the one or more processors 126, memory 128, communication equipment 130, etc. The power source 124 may be, as a non-limiting example, a power storage device (e.g., a battery, without limitation), a power inlet, a power regulator, or combination thereof.

In various examples, the handheld electronic computing device 106 is configured to communicate with the automated medicament delivery device 104 and the analyte sensor 102. The handheld electronic computing device 106 may be chosen from among a dedicated electronic device, a smart phone, a tablet computer, a wearable device (e.g., a smart watch, without limitation), a cloud computing device, and the like.

The handheld electronic computing device 106 may include one or more processors 132, memory 134 that stores instructions 136 to be executed by the one or more processors 132, communication equipment 138, and a user interface 140. The one or more processors 132 and memory 134 may be configured/programmed to perform any of the operations discussed above, as well as other control operations for managing the automated medicament delivery device 104 and the analyte sensor 102.

The communication equipment 138 is configured to facilitate communication (e.g., wireless communication, without limitation) of the handheld electronic computing devices 106 with other devices, such as the automated medicament delivery device 104 and the analyte sensor 102. The communication may be wired or wireless communication, such as via a wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In some of these examples, the automated medicament delivery device 104 and the handheld electronic computing devices 106 are paired via the short-range wireless protocol (e.g., paired via BLUETOOTH®, without limitation) and successful message transmissions between the automated medicament delivery device 104 and the handheld electronic computing devices 106 may be acknowledged.

The user interface 140 is configured to provide a user with information and obtain information from the user via one or more of a display, an audio speaker, an LED, a vibration motor, a button (e.g., a mechanical button, capacitive button, without limitation), a gesture-based interface, and the like.

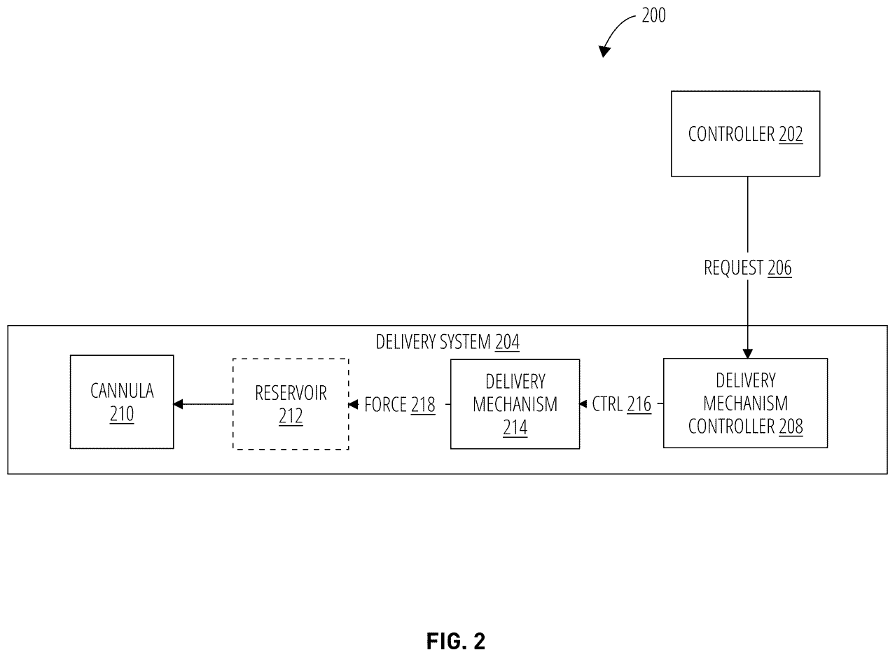

FIG. 2 is a block diagram of a medicament delivery system 200 for controlled administration of medicament to a user-body, in accordance with one or more examples.

The controller 202 is configured to manage the automated medicament delivery device 104 and, more generally, administration of medicament to a user-body. In one or more examples, controller 202 may be implemented by instructions 118 and one or more processors 126 of automated medicament delivery device 104 of FIG. 1. Furthermore, activity (e.g., determined and generated requests for delivery system 120, administered doses, etc.) of the automated medicament delivery device 104 and/or the medicament delivery system 200 may be stored in a memory.

In various examples, the controller 202 and the delivery system 204 may be realized in different devices (e.g., controller 202 may be realized in a physically different device (or devices) than delivery system 204 is realized, such as the handheld electronic computing device 106, without limitation), or in the same device. When realized in different devices, functionality of the controller 202 and the delivery system 204 may be implemented, at least in part, by respective memory and one or more processors of their respective devices. When realized in a same device, functionality of the controller 202 and the delivery system 204 may be implemented, at least in part, by like memory and like one or more processors, respective memory and respective one or more processors, or a combination thereof. Non-limiting examples of devices in which the controller 202, or a portion thereof, may be realized include: a handheld electronic computing device, such as a dedicated electronic device, a smart phone, a tablet computer, a wearable device (e.g., a smart watch, without limitation), a cloud computing device, and the like.

In various examples, the controller 202 may be configured to receive analyte data (e.g., from the analyte sensor 102, without limitation) including analyte values. In one or more examples, the controller 202 may determine information about analytes within a user-body at least partially based on analyte data, for example, amounts, trends, distributions, without limitation. The controller 202 may analyze information about analytes in a user-body and may present the information and/or analysis to a patient, caregiver, or healthcare provider, as a non-limiting example, via an application (e.g., executing on a personal computer, a smart phone, a cloud server, and/or combinations thereof).

In various examples, the controller 202 may be configured to receive information from inputs from the patient or a caregiver (e.g., when the patient ate a meal or when the patient exercised, without limitation), and inputs from other electronic devices (e.g., information from a smartwatch, without limitation) and to utilize such information as discussed herein. For example, in various examples, the controller 202 may utilize some or a totality of such information to determine amounts of medicament to administer and timing of administration of medicament. Further, controller 202 may also be configured to determine requests, including requests 206 to administer doses, and send those requests to the automated medicament delivery device 104.

In various examples, the controller 202 may be configured to determine a target dose amount to administer to a user of the medicament delivery system 200. The controller 202 may determine a target dose amount at least partially based on therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), a current glucose value, and derivatives thereof without limitation. The timing and target dose amounts associated with requests generated by controller 202 may be governed by one or more control algorithms, discussed below. For instance, the timing and target dose amounts associated with requests generated by controller 202 may be governed by one or more AID algorithms.

The controller 202 may send a request 206 to administer a dose to delivery system 204, and more specifically, delivery mechanism controller 208. The request 206 to administer a dose may include the target dose amount determined by the controller 202.

The cannula 210 is insertable into a user-body (e.g., with a tip thereof positioned subcutaneously, without limitation) and is configured to provide medicament to a user-body (e.g., subcutaneously into the user-body, without limitation).

The reservoir 212 is configured to store and retain a medicament therein. As a non-limiting example, the reservoir 212 may be a hollow body, a chamber, a vial, without limitation. In various examples, the reservoir 212 is a fluid reservoir for holding medicament and may be, as a non-limiting example, formed from the walls of a cartridge. In the cartridge example, the delivery system 204 may include a chamber (i.e., a space or region defined within the delivery system 204) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in the reservoir 212 will be greater in a pre-filled state than the volume in an exhausted state. Additionally or alternatively to the cartridge example, the delivery system 204 is a multi-part delivery device where one of the two parts includes the reservoir 212 and the other one of the two parts includes the delivery mechanism controller 208. The other one of the two parts may optionally further include the controller 202. Either one of the two parts may optionally include the delivery mechanism 214 (e.g., a pump mechanism, without limitation). The one of the two parts that includes the reservoir 212 is disposable (i.e., a “disposable part”) and configured to be removable secured to the other part of the medicament delivery system 200. When the reservoir 212 is exhausted, the disposable part may be removed and a replacement part including a reservoir 212 optionally in a pre-filled state.

The delivery mechanism 214 is configured to urge fluid in the reservoir 212 toward an interface for dispensing fluid (interface not shown). In various examples, the delivery mechanism 214 may be positioned adjacent to the reservoir 212. The delivery mechanism 214 is configured to cause an amount of the medicament to be administered to the user-body by causing the amount to flow from the reservoir 212 toward and into a user-body via cannula 210, which is in fluidic communication with the reservoir 212. In various examples, the delivery mechanism 214 may utilize any suitable mechanism to generate positive displacement or negative displacement to transfer amounts of medicament from the reservoir 212 toward the cannula 210 and a user-body. Non-limiting examples of mechanisms include a ratchet gear pump, a peristaltic pump, a linear peristaltic pump, a piston pump, a gear pump, a bellows pump, or a diaphragm pump.

For example, the delivery mechanism 214 may apply a force to an urging mechanism (e.g., a plunger, flexible-walled tube, without limitation) free to move within the reservoir 212, and via such a force, move the urging mechanism in a direction that urges fluid in the reservoir 212 toward the aforementioned interface. In one or more examples, the delivery mechanism 214 may include an electrical motor (e.g., an AC or DC motor) that produces a force to, directly or indirectly, move the urging mechanism to perform a delivery action. A delivery action dispenses at a predetermined rate (i.e., a predictable amount of fluid over a predictable duration of time). The delivery mechanism 214 may be capable of multiple rates of delivery, and in one or more examples, may be preconfigured to use a same rate of delivery all the time, or, in some cases, may be provided discretion to determine a rate of delivery consistent with a target dose amount included with a request 206.

Such an electric motor may be a current controlled electric motor, voltage controlled electric motor, pulse-width controlled electric motor, or combination or sub combination thereof. Such an electronic motor may be directly or indirectly digitally controlled. A control signal 216 may be determined and generated by the delivery mechanism controller 208 to correspond to a delivery action. The control signal 216 may also be referred to herein as a “command 216” or an “instruction 216.”

The delivery mechanism controller 208 may generate control signals 216 corresponding to one or more delivery actions at least partially based on a request 206 to administer a dose received from the controller 202 or elsewhere (e.g., the handheld electronic computing device 106). The control signal 216 may include first control signals to cause the delivery mechanism 214 to generate a resultant force 218, and a second, different control signal to cause drive delivery mechanism 214 to not or stop generating the force 218. Utilizing control signals 216, the delivery mechanism controller 208 may control a length of a duration of time that the delivery mechanism 214 produces the force 218 and applies it to dispense fluid from the reservoir 212, and indirectly, an amount of fluid dispensed from the reservoir 212.

When delivery mechanism controller 208 generates the control signal 216 in response to a request 206 to administer a dose from the controller 202 or elsewhere, it may generate the control signal 216 at least partially based on a value of a target dose amount included with, or indicated by, the request 206 to administer a dose. One or more delivery actions may be utilized to dispense an amount fluid corresponding to a dose amount determined by the controller 202. For example, a fluid amount dispensed according to a delivery action may be less than a dose amount. Generally speaking, the delivery mechanism 214, and the delivery system 204, are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more examples, the delivery mechanism controller 208 may be configured to determine and generate feedback information about delivery actions, such as times of delivery actions and dispensed amounts, without limitation. Feedback information may be generated based on information generated by the delivery mechanism 214 or by sensors utilized by the delivery mechanism controller 208 to monitor operation of the delivery mechanism 214 (sensors not depicted). For example, sensors to monitor mechanical movement, current consumption, a voltage profile of an electric motor, without limitation. Such information may be logged and provided to and stored at the controller 202 and/or the handheld electronic computing device 106, without limitation, for e.g., later processing or reading, without limitation. For example, the logs can be processed to determine patterns that may be utilized to determine whether the delivery system 204 is operating as expected (e.g., in a predictable manner, without limitation), and if a difference between actual and expected operation exceeds a threshold, the delivery mechanism controller 208 may be updated (e.g., firmware, parameters, or both, of the delivery mechanism controller 208 may be updated, without limitation) to compensate or correct for the difference. Additionally or alternatively to updating the firmware or parameters, in a multi-part system, one or more parts including the delivery mechanism controller 208 or the delivery mechanism controller 208 may be indicated as needing replacement (e.g., an alarm or alert is generated at the delivery system 204, the medicament delivery system 200, a mobile device, and/or computer in communication therewith, without limitation).

### Control Algorithm Architecture

As noted above, values of target dose amounts and timing of requests to administer the target dose amounts generated by the controller 202 may be governed by one or more control algorithms (AID algorithms) implemented at the controller 202. Generally speaking, such a control algorithm may, via one or more control actions, try to cause an amount of analyte in the body (represented by values captured by, or at least partially based on, an analyte sensor or monitor, without limitation) to track (e.g., at least substantially match) a target amount of analyte (in control terms, the target amount of analyte is the “set point”) in the body. The control actions may include an amount and a timing of administration of doses of medicament that functions as a therapeutic agent in the body.

In one or more examples, a control algorithm may employ a modular design in which core functionality may be separated from dependent functionality. Dependent functionality includes, as non-limiting examples, functionality that may be implementation-specific to a current environment, such as software abstraction for an analyte sensor. Such dependent functionality may include software services which interface with implementation-specific features that affect inputs or outputs to the control algorithm. Dependent functionality may include, as a non-limiting example, functionality for managing algorithm initialization and upload of administration history, managing the control algorithm's state and data therapy variables, and maintaining cycle-to-cycle data utilized by the algorithm such as analyte values, current or historical. Dependent functionality may include functionality responsible for sending requests to administer doses to delivery system 204 which are determined by the control algorithm.

Transmission of data, including without limitation, a request 206 to administer a dose, may occur over wired, wireless, or a combination thereof communication paths, in a synchronous or asynchronous manner. In one or more examples, the control algorithm may include one or more layers to provide safety or other operational constraints (e.g., for edge case handling, without limitation).

In one or more examples, a control algorithm may determine a target dose amount to include within a request at least partially based on a dynamic model of a user-body's response, in terms of amount of analyte in the user-body, to administration of analyte to the user-body. The control algorithm may determine a future amount of analyte or a change in amount of analyte over a predetermined duration of time for a respective dose amount and compare the determined future amount or change to a target amount or change. The control algorithm may determine target dose amounts according to control intervals that occur according to a predetermined schedule, on-demand, or both. In one or more examples, the control intervals may correspond to diurnal intervals such as day-night, weeks, days, twenty-four (24) hours, single hours, and sub-intervals of the same, such as 5-minute intervals.

In some cases, the control algorithm may be or include a control algorithm that handles constraints, such as a model-predictive-control (MPC) algorithm. Non-limiting examples of constraints include: upper and lower bounds on analyte levels can be set to prevent dangerous hypo- or hyperglycemia; medicament delivery rates capable by delivery system 204 can be constrained to prevent over- or under-dosing; and considerations related to medicament-on-board to, e.g., prevent stacking of medicament doses waiting to work on analyte in the body (e.g., stacking of insulin waiting to work on glucose, without limitation).

One or more examples discussed herein may refer to administering medicament or a medicament therapy to a user or the user-body. Such discussion is intended to encompass examples where medicament or a medicament therapy is administered to a user by automated medicament delivery devices discussed herein, examples where requests to administer doses in accordance with administering medicament or medicament therapy to the user or user-body are generated by a controller and sent to a delivery device, and examples where instructions (e.g., control signals, without limitation) in accordance with dose amounts and timing included with such requests to administer doses are generated by a delivery mechanism controller and sent to a delivery mechanism.

Identifying current behavior trends of users of AID systems and predicting future behavior trends and/or events based on identified behavior trends of those users can enhance a glycemic performance of the AID systems (e.g., how well the AID systems manage and maintain a user's blood glucose levels within a target range over a period of time). As used herein, the term “behavior trend” may refer to a behavior inferred from identified patterns in collected therapy data (e.g., CGM data and/or insulin-on-board (IOB) data) over a given period of time. In particular, behavior trends may refer to inferred behaviors associated within a specific time of day and/or a specific time of a specific day of the week. For example, behavior trends may refer to behaviors, such as, bolus events, bolus dosing amounts relative to current analyte levels and/or IOB, bolus dosing aggressiveness, meal events, meal sizes, meal carb counts, exercise events, exercise rigors, and relationships (e.g., timings) between any of the foregoing behaviors. Furthermore, one or more behavior trends may include an associated range of time within a day and/or a range of time within a specific day of the week. In some embodiments, a behavior trend may include an associated probability of the behavior trend occurring during a given day within a range of time and/or during a specific day of the week within a range of time.

One way to gain insights into future behavior is by leveraging historical patterns or habits (e.g., historical behavior trends). Identifying historical behavior trends can provide information about possible future behavior trends and/or events of a given user. As a non-limiting example, consider the habitual timing of breakfast for many individuals, which typically occurs around 8:00 AM under ordinary circumstances, and that habit will likely continue unless unusual situations arise. Similarly, some people may have a consistent routine of going to the gym from 2:00 PM to 3:00 PM. The foregoing patterns of life can be identified and utilized to inform the AID systems by indicating that for a given user, there is a high likelihood that the user will eat a meal and/or visit the gym at a certain time in upcoming days. Furthermore, based at least partially on the identified behavior trends and future events predicted from the identified behavior trends, operation of the AID systems can be adjusted to improve the glycemic performance of the AID systems.

Improving glycemic performance of the AID systems also improves the user's quality of life by reducing the need for frequent interactions with the AID systems. For example, the AID system can automatically identify behavior trends of the user and adapt (e.g., automatically adapt) operation of the AID based on these identified behavior trends. Additionally, the identified behavior trends can also be implemented to predict glucose levels while using machine learning algorithms and/or utilized in decision-making processes for AID systems.

Statistical Features from Therapy Data:

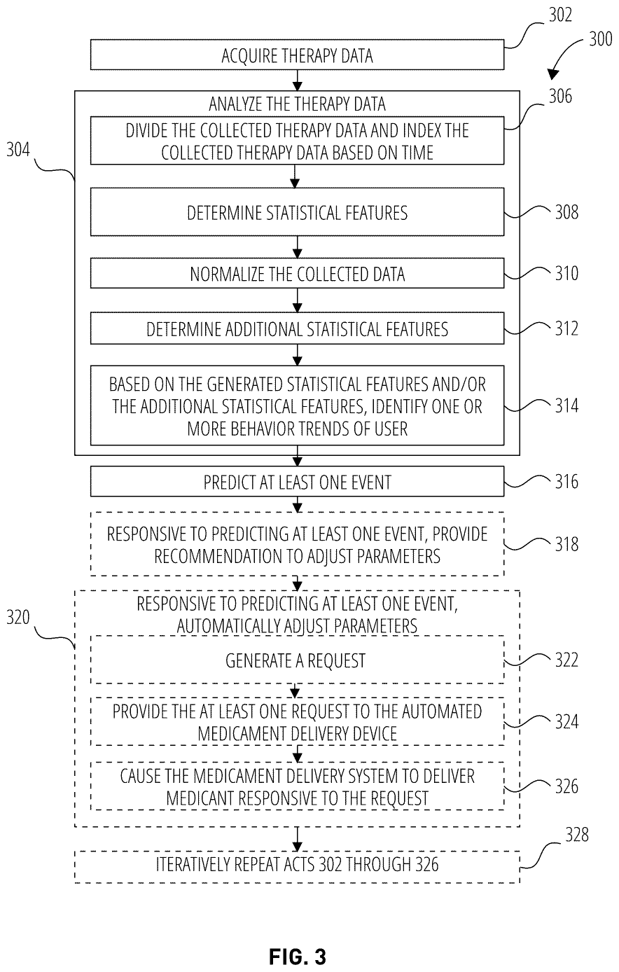

FIG. 3 shows a flowchart of a method 300 of managing medicament therapy for one or more users. In some embodiments, one or more acts of the method 300 may be performed and/or executed by the controller 202 of the medicament delivery system 200. However, the disclosure is not so limited, and one or more acts of the method 300 may be performed, executed, and/or initiated by the delivery mechanism controller 208 of the delivery system 204, other controllers of the system 100, the handheld electronic computing device 106, and/or one or more other remote devices. For purposes of the present disclosure, the acts of the method 300 are described as being performed, executed, and/or initiated by the controller 202 of the medicament delivery system 200.

The method 300 may include acquiring and collecting therapy data associated with a given user, as shown in act 302 of FIG. 3. For example, the controller 202 of the medicament delivery system 200 may acquire the therapy data associated with the given user. In some embodiments, acquiring the therapy data may include acquiring therapy data for at least a previous seven-day period. In additional embodiments, acquiring the therapy data may include acquiring the therapy data for at least a previous fourteen-day period, a thirty-day period, a sixty-day period, or a longer time period. For the embodiments described herein, a seven-day period is utilized for ease of description; however, it is understood that any time period may be utilized.

In some embodiments, the time period may be a moving time period. For instance, the time period may be a most-recent seven-day period. In some embodiments, the time period moves (e.g., is updated) every twenty-four hours. In additional embodiments, the time period is constantly changing in real-time and includes a most-recent 168-hour period.

In some embodiments, the therapy data may include one or more of continuous glucose monitoring (CGM) data (e.g., analyte levels) captured during the previous seven-day period and insulin-on-board data (e.g., an ongoing amount of active rapid-acting insulin in the user's body) for the previous seven-day period. The IOB data may include a calculated amount of remaining insulin from previously administered bolus doses multiplied by the insulin sensitivity factor (ISF) and/or a correction factor over the previous seven-day period. In some embodiments, the controller 202 may receive the CGM data from the analyte sensor 102. Additionally, the controller 202 may determine the IOB data from tracked administered bolus doses, either administered and tracked by the medicament delivery system 200 or manually recorded by the user via the medicament delivery system 200 and/or the handheld electronic computing device 106. In view of the foregoing, the therapy data may include measured CGM data (i.e., analyte levels) and/or determined IOB data over the seven-day period.

Responsive to collecting the therapy data, the method 300 may include analyzing the therapy data to identify one or more behavior trends represented in the therapy data, as shown in act 304 of FIG. 3. For example, the controller 202 may analyze the therapy data. In one or more embodiments, analyzing the therapy data may include identifying one or more behavior trends represented in a previous at least seven-day period.

In some embodiments, analyzing the therapy data may include dividing the collected therapy data into distinct datasets and indexing the therapy data based on a correlating time of day, as shown in act 306 of FIG. 3. For example, the controller 202 may divide the collected therapy data into distinct datasets and index the therapy data based on a correlating time of date. In some embodiments, dividing the collected therapy data may include dividing the collected therapy data into seven distinct datasets, each dataset representing a 24-hr period of time (e.g., a day). In some embodiments, a “24-hr period of time” may refer to a period of time from 12:00 AM to 12:00 PM. In other embodiments, a “24-hr period of time” may refer to any other continuous period of 24 hrs. In one or more embodiments, each dataset includes 288 data points representing a 24-hr period of time with five-minute sampling intervals (e.g., a data point every five minutes).

Indexing the therapy data may include creating a data structure (e.g., a B-tree or hash table) that indexes (e.g., labels, correlates) each data point of each dataset with a time of day at which the data was captured and/or a time of day for which the data point reflects an analyte value and/or determined IOB for the user. Indexing the therapy data based on represented time of day enables accessing data of the therapy data that corresponds to specific times of day (e.g., meal times).

Additionally, in some embodiments, indexing the therapy data may further include aligning (e.g., correlating) the data points of the distinct datasets of the therapy data based on times of day represented by the data points. For example, the controller 202 may align the data points of the distinct datasets of the therapy data based on times of day represented by the data points. For instance, each given data point of each distinct dataset may be correlated with data points of the other distinct datasets representing a same time of day as the given data point. As a non-limiting example, each data point representing the time of day of 12:05 μm of each distinct dataset may be aligned (e.g., correlated).

Analyzing the therapy data may further include determining statistical features from the divided and aligned therapy data, as shown in act 308 of FIG. 3. For example, the controller 202 may determine statistical features from the divided and aligned therapy data. In some embodiments, the statistical features may include mean values. As a non-limiting example, for each time of day represented by the 288 data points of each distinct dataset, a mean value may be determined. In other words, a mean value may be determined for a given time of day across all seven days represented by the distinct datasets. Put yet another way, for each time of day represented by the data points of each distinct dataset, a mean value of the respective data points of the distinct datasets associated with the given time of day may be determined. As a result, the determined mean data consists of 288 data points, with each data point corresponding to a specific 5-minute interval of the day.

Additionally, analyzing the therapy data may include normalizing the data points of the therapy data, as shown in act 310 of FIG. 3. For example, the controller 202 may normalize the data points of the therapy data. In some embodiments, normalizing the data points of the therapy data may include normalizing the mean data determined in act 308 of FIG. 3. In additional embodiments, normalizing the data points of the therapy data may may include normalizing each data point of each distinct dataset of the therapy data (e.g., the therapy data in a state as determined in act 306 of FIG. 3).

In some embodiments, each data point is normalized to a range of 0 to 1. In one or more embodiments, each data point may be normalized via a min-max normalization technique. The min-max normalization technique rescales the values of each data point so that the values fall within the range of 0 to 1. The formula for min-max normalization is shown in Equation 1:

$$𝓏 = \frac{x - min ( x )}{max ( x ) - min ( x )} \qquad Equation 1$$

Where (z) is the normalized value, (x) is the original value of a given data point, (min (x)) is the minimum value in the therapy data, and (max (x)) is the maximum value in the therapy data. As noted above, in some embodiments, the mean data of the therapy data determined in act 308 may be normalized and the maximum and minimum values of the mean data may be utilized in Equation 1. In additional embodiments, the therapy data, as divided and indexed above in act 306, may be normalized and the maximum and minimum values of the therapy data as acquired in act 302 may be utilized in Equation 1. The normalized values may provide probabilities of behavior trends (described below) occurring at a specific time of day and/or at a specific time of a specific day of the week. As a non-limiting example, normalized values approaching 1 may indicate a high probability of behavior trends associated with a blood glucose increase, such as meal intake. Conversely, normalized values approaching 0 may indicate a high probability of behavior trends associated with a blood glucose decrease, such as exercise and/or physical activity.

Additionally, the method 300 may include determining additional statistical features from the divided and aligned therapy data, as shown in act 312 of FIG. 3. For example, the controller 202 may determine the additional statistical features from the divided and aligned therapy data. In some embodiments, the additional statistical features may include one or more of a first (1st) quartile of the therapy data, a second (2nd) quartile of the therapy data, a third (3rd) quartile of the therapy data, a maximum value of the therapy data, a minimum value of the therapy data, a range between the maximum value and the minimum value of the therapy data, a standard deviation of the therapy data, a differentiation of the therapy data, etc. Each feature may individually or collectively correspond to a section of the target populations utilizing the therapy for whom the outcome of the therapy is or is not desirable. Thus, the features may be correlated to identify the target populations that are experiencing negative or most negative outcomes, and drive them to the features shared by the target populations experiencing positive of most positive outcomes.

The first (1st) quartile of the therapy data may include a value below which 25% of the therapy data falls. In particular, it is the median of a lower half of the therapy data. The first (1st) quartile of the therapy data may be calculated by arranging the therapy data in ascending order and determining the median of a lower (first) half of the therapy data.

The second (2nd) quartile of the therapy data may include a middle value of therapy data below which 50% of the therapy data falls. The second (2nd) quartile of the therapy data may be calculated by arranging the therapy data in ascending order and determining the middle value of the therapy data.

The third (3rd) quartile of the therapy data may include a value below which 75% of the therapy data falls. In particular, it is the median of a upper half of the therapy data. The third (3rd) quartile of the therapy data may be calculated by arranging the therapy data in ascending order and determining the median of an upper (second) half of the therapy data.

The maximum value may include the largest value of the therapy data, and the minimum value may include the smallest value in the therapy data. The range may include the difference between the maximum value and the minimum value. The standard deviation may include a measure of an amount of variation or dispersion of the therapy data. The standard deviation may be determined via any conventional method. For example, the standard deviation can be determined by finding the mean (average) of the therapy data, subtracting the mean from each data point and square the difference, finding the average of the squared differences, and taking the square root of the average of the squared differences.

The differentiation may include finding the derivative of one or more functions represented (e.g., curves) by the therapy data. The derivative may represent a rate at which the function (e.g., the therapy data) is changing at any given point through the period of time. Furthermore, the differentiation may enable finding a slope of the function and optimizing functions.

The method 300 may further include, based at least partially on the determined statistical features, the normalized therapy data, and/or the additional statistical features, identifying one or more behavior trends represented in the seven-day period of the user, as shown in act 314 of FIG. 3. For example, the controller 202 may identify one or more behavior trends of the user based at least partially on the determined statistical features, the normalized therapy data, and/or the additional statistical features. In some embodiments, the behavior trends may be associated with particular times of day and/or particular times of day of specific days of a week and may have an associated probability. In one or more embodiments, the behavior trends may include one or more of bolus dosing times, bolus dosing amounts, bolus aggressiveness, meal intake times, meal sizes, exercise times, exercise rigor, etc.

As used herein, the term “bolus aggressiveness” may refer to how quickly and in what amount a bolus dose is administered. Furthermore, the “bolus aggressiveness” may refer to how quickly and in what amount a bolus dose is administered when considering how far off the user's blood glucose level is from a target range.

As used herein, the term “exercise rigor” may refer to a strictness, an intensity, and a thoroughness with which an exercise regimen is followed.

As a non-limiting example, analyzing the therapy data may include identifying at least one of a peak or a dip indicated by the determined mean values, and based at least partially on peaks and dips represented in the mean data determined in act 308 of FIG. 3, consistent meal intake times, exercise times, and bolus dosing times may be identified. Furthermore, the normalized data associated with identified meal intake times, exercise times, and bolus dosing times may provide a probability rate of the meal intake, exercise, and bolus dosing occurring on a given day. Furthermore, slopes (e.g., rates of change) of the spikes and dips may indicate exercise rigors, bolus aggressiveness, and/or consistency of meal intake times, exercise times, and bolus dosing times. As non-limiting examples, steeper slopes may indicate one or more of more consistent timing of events (e.g., within the mean data), aggressiveness of bolus doses (e.g., within the therapy data and/or the mean data), and rigor of exercise (e.g., within the therapy data and/or the mean data).

Furthermore, as noted above, behavior trends may include relationships between identified events (e.g., meal intakes, exercise, bolus dosing). For instance, a behavior trend may include timings between events. As used herein, the term “event” may refer to a meal event (i.e., meal intake), an exercise event (i.e., performance of exercise), and/or a bolus dose (i.e., delivery of a bolus dose). In some instances, the term “event” may refer to a value of a measurement and/or calculation of a parameter (e.g., CGM value, IOB value, etc.) at a given time.

As noted above, the behavior trends may be associated with particular times of day and/or particular times of day of specific days of a week. Accordingly, the behavior trends may indicate typical meal times, exercise schedules, changes in behavior on weekends relative to weekdays, periodic hormonal variations such as menstrual cycles, recurring events such as job-related periodic travels, and/or psychological stress that tends to occur periodically. In view of the foregoing, identifying at least one behavior trend further may include identifying at least one of a time of day or a specific day of week and a time of day associated with an identified at least one of a meal event, an exercise event, or a bolus dose. Furthermore, in view of the foregoing, identifying at least one behavior trend may further include identifying at least one of a meal size, an exercise rigor, or a bolus dosing aggressiveness.

The method 300 may further include, based at least partially on the identified behavior trends, predicting at least one event, as shown in act 316 of FIG. 3. For example, the controller 202 may predict the at least one event. In some embodiments, predicting the at least one event may include predicting at least one of a meal event, an exercise event, or a bolus dose. Furthermore, predicting the at least one event may include predicting at least one of a meal size, an exercise rigor, or a bolus dosing aggressiveness. Moreover, predicting the at least one event may include predicting at least one of a predicted time of day or a predicted time of day and day of the week for the predicted event to occur.

As non-limiting examples, if the identified behavior trends indicate that an event has historically occurred within a given range of time on a given day of the week and/or daily, act 316 may include predicting that the event will occur during a same range of time on a same day of the week and/or daily. Furthermore, depending on the type of event, the probability of the event occurring, which may be determined in act 310 of FIG. 3, may be associated with the predicted event. In some embodiments, the method 300 may only predict events for which the behavior trends indicate that the event has historically occurred above a threshold incidence rate. As used herein, the term “incidence rate” may refer to a measure of a frequency and regularity at which the behavior trends indicate that the event has historically occurred. For instance, the method 300 may include only predict events having a historical incidence rate of occurring over 50% of the time. In additional embodiments, the method 300 may include only predicting events having a historical incidence rate of occurring over 60%, 70%, 80%, or 90% of the time.

In alternative embodiments, the method 300 may include predicting at least substantially all of events indicated by the behavior trends; however, the method 300 may include associating a determined incidence rate with the predicted events.

Responsive to predicting the at least one event, the method 300 may optionally include generating and providing a recommendation to the user to adjust one or more parameters utilized in administering insulin to the user, as shown in act 318 of FIG. 3. For example, the controller 202 of the medicament delivery system 200 may generate a recommendation to adjust the one or more parameters and cause the recommendation to be provided to the user. In some embodiments, the controller 202 may cause the recommendation to be displayed on the user interface 140 of the handheld electronic computing device 106. In additional embodiments, the controller 202 may cause the recommendation to be displayed on a user interface of the automated medicament delivery device 104 and/or the medicament delivery system 200.

The one or more parameters may include amounts of bolus doses, recommended bolus dose timings, carb count of meals, basal rates, insulin-to-carbohydrate ratios, insulin sensitivity/correction factors, active insulin times, target blood glucose levels, and total daily insulin doses.

As a non-limiting example, responsive to predicting an event that will increase blood glucose levels (e.g., a meal intake) within a next relatively short period of time (e.g., 3 hrs, 2 hrs, 1 hr, 30 minutes), the controller 202 may generate a recommendation to adjust a basal rate and/or timing or amount of a bolus dose in anticipation of the event. For instance, the controller 202 may generate a recommendation to adjust a basal rate and/or a bolus dose to enable early deliver of insulin in anticipation of the event and better Time in Range (TIR) control. Likewise, responsive to predicting an event that will decrease blood glucose levels (e.g., exercise) within a next relatively short period of time (e.g., 3 hrs, 2 hrs, 1 hr, 30 minutes), the controller 202 may generate a recommendation to adjust a basal rate and/or timing or amount of a bolus dose and adopt a more conservative insulin delivery regime prior to the event. A conservative approach may include lower initial doses, making incremental adjustments, and avoiding aggressive corrections in a time leading up to the forthcoming event. For instance, the controller 202 may generate a recommendation to adjust a basal rate and/or a bolus dose to reduce an amount insulin delivered prior to the event and reduce a risk of exercise-related hypoglycemia.

Referring still to act 318, in some embodiments, the method 300 may require user input to change the parameters. For example, the user made have to manually change the parameters within the memory 128 of the automated medicament delivery device 104 (e.g., within settings of the automated medicament delivery device 104), or the user may have to manually change the parameters in another application on, for example, the handheld electronic computing device 106 or on another device. In additional embodiments, the controller 202 of the medicament delivery system 200 may change the parameters with the memory 128 of the automated medicament delivery device 104 responsive to an user input indicating acceptance of the recommendation by the user.

In some embodiments, responsive to predicting the at least one event, the method 300 may optionally include automatically adjusting parameters utilized in administering insulin to the user, without user input, as shown in act 320 of FIG. 3. For example, the controller 202 of the medicament delivery system 200 may automatically adjust parameters utilized in administering insulin to the user. In other words, the controller 202 may automatically adjust operation of the medicament delivery system 200.

As a non-limiting example, responsive to predicting an event that will increase blood glucose levels (e.g., a meal intake) within a next relatively short period of time (e.g., 3 hrs, 2 hrs, 1 hr, 30 minutes), the controller 202 may automatically adjust a basal rate and/or timing or amount of a bolus dose in anticipation of the event. For instance, the controller 202 may automatically adjust a basal rate and/or a bolus dose to enable early deliver of insulin in anticipation of the event and better Time in Range (TIR) control. Likewise, responsive to predicting an event that will decrease blood glucose levels (e.g., exercise) within a next relatively short period of time (e.g., 3 hrs, 2 hrs, 1 hr, 30 minutes), the controller 202 may automatically adjust a basal rate and/or timing or amount of a bolus dose and adopt a more conservative insulin delivery regime prior to the event. For instance, the controller 202 may automatically adjust a basal rate and/or a bolus dose to reduce an amount insulin delivered prior to the event and reduce a risk of exercise-related hypoglycemia.

In some embodiments, automatically adjusting parameters utilized in administering insulin to the user may include generating at least one request 206 to adjust administration of insulin to the user (e.g., adjust operation of the automated medicament delivery device 104) according to the adjusted parameters, as shown in act 322. Furthermore, in some embodiments, generating the at least one request 206 and act 322 may occur responsive to a user approving the recommendation to adjust one or more parameters described above in regard act 318 of FIG. 3. In one or more embodiments, the controller 202 may generate the at least one request 206. In some embodiments, the request 206 may include instructions to administer basal dosing and/or a bolus dose based on the adjusted parameters.

Additionally, automatically adjusting parameters utilized in administering insulin to the user may include providing the generated at least one request to the automated medicament delivery device 104, as shown in act 324 of FIG. 3. For instance, the controller 202 may provide the at least one request 206 to the delivery mechanism controller 208 of the delivery system 204 of the medicament delivery system 200 via any of the manners described above in regard to FIG. 1 and FIG. 2.

Moreover, the method 300 may optionally include causing the medicament delivery system 200 to deliver insulin to the user's body in accordance with the request 206, as shown in act 326 of FIG. 3. For instance, the method 300 may include causing the medicament delivery system 200 to deliver insulin to the user's body responsive to the request 206 via any of the manners described above in regard to FIG. 1 and FIG. 2. Put another way, the method 300 may include adjusting operation of one or more of the medicament delivery system 200 and/or automated medicament delivery device 104 and causing the medicament delivery system 200 and/or automated medicament delivery device 104 to deliver insulin according to the adjusted operation.

Furthermore, the method 300 may optionally include iteratively repeating act 302 through act 326, as shown in act 328 of FIG. 3. For example, the controller 202 may iteratively repeat act 302 through act 326. In some embodiments, act 302 through act 326 may be repeated every 24 hours. In other embodiments, the act 302 through act 326 may be repeated every 12 hours, 6 hours, 3 hours, 1 hour, 30 minutes, or any other period of time.

Repeating act 302 through act 326 may enable the medicament delivery system 200 to identify new behavior trends and/or changing behavior trends (e.g., a new exercise regime or a new diet). As a result, the medicament delivery system 200 may consistently be analyzing a previous seven-day period using a moving window approach.

### Time-Based Features:

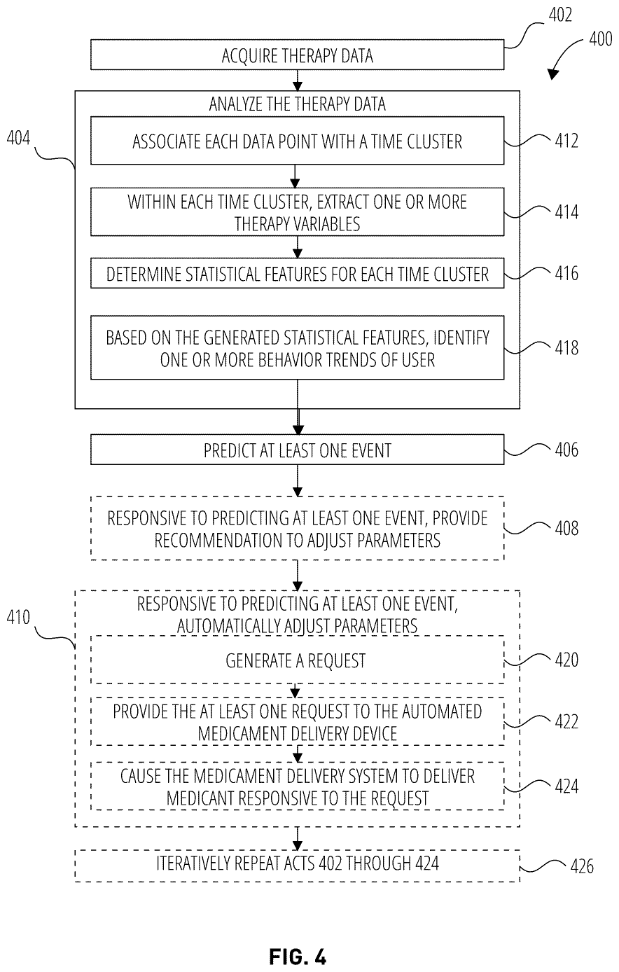

FIG. 4 shows a flowchart of a method 400 of managing medicament therapy for one or more users. In some embodiments, one or more acts of the method 400 may be performed and/or executed by the controller 202 of the medicament delivery system 200. However, the disclosure is not so limited, and one or more acts of the method 400 may be performed, executed, and/or initiated by the delivery mechanism controller 208 of the delivery system 204, other controllers of the system 100, the handheld electronic computing device 106, and/or one or more other remote devices. For purposes of the present disclosure, the acts of the method 400 are described as being performed, executed, and/or initiated by the controller 202 of the medicament delivery system 200.

The method 400 may include acquiring and collecting therapy data associated with a given user, as shown in act 402 of FIG. 4. For example, the controller 202 of the medicament delivery system 200 may acquire the therapy data associated with the given user. In some embodiments, acquiring the therapy data may include acquiring therapy data for at least a previous seven-day period. In additional embodiments, acquiring the therapy data may include acquiring the therapy data for at least a previous fourteen-day period, a thirty-day period, a sixty-day period, or a longer time period. For the embodiments described herein, a seven-day period is utilized for ease of description; however, it is understood that any time period may be utilized.

In some embodiments, the time period may be a moving time period. For instance, the time period may be a most-recent, seven-day period. In some embodiments, the time period moves (e.g., is updated) every twenty-four hours. In additional embodiments, the time period is constantly changing in real-time and includes a most-recent 168-hour period.

In some embodiments, the therapy data may include one or more of continuous glucose monitoring (CGM) data (e.g., analyte levels) captured during the previous seven-day period and insulin-on-board (IOB) data (e.g., an ongoing amount of active rapid-acting insulin in the user's body) for the previous seven-day period. The IOB data may include a calculated amount of remaining insulin from previously administered bolus doses multiplied by the insulin sensitivity factor (ISF) and/or a correction factor over the previous seven-day period. In some embodiments, the controller 202 may receive the CGM data from the analyte sensor 102. Additionally, the controller 202 may determine the IOB data from tracked administered bolus doses, either administered and tracked by the medicament delivery system 200 or manually recorded by the user via the medicament delivery system 200 and/or the handheld electronic computing device 106. In view of the foregoing, the therapy data may include measured CGM data (i.e., analyte levels) and/or determined IOB data over the seven-day period.

Responsive to collecting the therapy data, the method 400 may include analyzing the therapy data to identify one or more behavior trends represented in the therapy data, as shown in act 404 of FIG. 4. For example, the controller 202 may analyze the therapy data.

In some embodiments, analyzing the therapy data may include associating each data point of the therapy data with a time cluster, as shown in act 412 of FIG. 4. For example, the controller 202 may associate each data point of the therapy data with a time cluster. In some embodiments, each data point of the therapy data may be associated with a time cluster based on a timestamp of the given data point (e.g., a time of day at which the given data point was captured and/or the time of day for which the given data point represents).

In one or more embodiments, each time cluster represents a thirty-minute (30-minute) time interval (i.e., window) of a day. For instance, a given day may include forty-eight (48) time clusters. As a non-limiting example, a first time cluster of a day may represent a time interval from 12:00 AM to 12:30 AM, while a last time cluster of the day may represent 11:30 μm to 12:00 am. As a result, each data point captured or representing a time of day within a given time cluster is associated with the given time cluster. As a non-limiting example, all data points across all seven days of the therapy data captured within a time interval from 12:00 AM to 12:30 AM will be associated with the time cluster (i.e., a single time cluster) corresponding to the time interval from 12:00 AM to 12:30 AM. Furthermore, while the time clusters are described herein as representing thirty-minute (30-minute) intervals, the disclosure is not so limited, and the time clusters may represent any time intervals (e.g., 1-hr intervals, 2-hr time intervals, etc.).

In some embodiments, the therapy data may be associated with time clusters via fixed interval clustering techniques. In one or more embodiments, the data points of the therapy data are tagged or labeled with their respective time cluster. In additional embodiments, the data points of the therapy data are not tagged or labeled but are grouped based on timestamps.

Responsive to associating each data point with a time cluster, the method 400 may include extracting, from the therapy data (e.g., the data points) within each time cluster and/or the therapy data as a whole, one or more therapy variables, as shown in act 414 of FIG. 4. For example, the controller 202 may extract, from the therapy data (e.g., the data points) within each time cluster and/or the therapy data as a whole, one or more therapy variables. In particular, extracting one or more therapy variables may include inspecting and/or filtering the therapy data via any known data extraction methods. In one or more embodiments, extracting one or more therapy variables may include extracting one or more of CGM values, derivatives of the CGM values, meal insulin values, algorithm-derived insulin values, correction IOB values, TDI (Total Daily Insulin) values, meal IOB values, algorithm IOB values, and total IOB values represented within each time cluster.

The CGM values may include measurements from the analyte sensor 102. The derivative of CGM value may represent how quickly the CGM values are changing over time. The meal insulin values may represent an amount of insulin administered to address carbohydrates consumed via meals. The algorithm-derived insulin values may represent insulin dose determined via one or more algorithms to manage blood glucose levels. The correction IOB values may represent amounts of insulin still active in a user's body from correction doses. The TDI includes basal doses automatically delivered to the user-body via the automated medicament delivery device 104 according to an AID algorithm during the 24-hr period and all bolus doses administered during the 24-hr period. The meal IOB values may represent insulin still active in the body from insulin doses administered responsive to meal consumption. The algorithm IOB values may represent amounts of insulin still active in the user's body from insulin doses calculated by one or more algorithms. The total IOB values may include a total amount of insulin still active in the user's body from all sources.

Analyzing the therapy data may further include, for each time cluster of therapy data, determining statistical features for each of the one or more therapy variables, as shown in act 416 of FIG. 4. For example, the controller 202 may determine statistical features for each of the one or more therapy variables of the time clusters of therapy data. In some embodiments, determining the statistical features may include, for each of the one or more therapy variables (i.e., for the data points associated with each of the one or more therapy variables), determining one or more of a mean value, a first (1st) quartile, a second (2nd) quartile, a third (3rd) quartile, a maximum value, a minimum value, a range between the maximum value and the minimum value, a standard deviation, and/or a differentiation of the data points associated with each of the one or more therapy variables. The foregoing statistical features may be determined via any of the manners described above in regard to FIG. 3; however, in some embodiments, the statistical features may be individually determined for each of the one or more therapy variables of each time cluster determined above in regard to act 414 of FIG. 4.

Referring still to act 416 of FIG. 4, the determined statistical features may collectively represent characteristics of the 30-minute intervals within each cluster.

The method 400 may further include, based at least partially on the determined statistical features, identifying one or more behavior trends represented in the seven-day period of the user, as shown in act 418 of FIG. 4. For example, the controller 202 may identify one or more behavior trends of the user based at least partially on the determined statistical features. In some embodiments, the behavior trends may be associated with particular times of day and/or particular times of day of specific days of a week and may have an associated frequency. In one or more embodiments, the behavior trends may include one or more of bolus dosing times, bolus dosing amounts, bolus aggressiveness, meal intake times, meal sizes, exercise times, exercise rigors, etc. Furthermore, the behavior trends may include any of the behavior trends described above in regard to FIG. 3.

As a non-limiting example, based at least partially on the determined means of CGM values within the time clusters, changes (e.g., peaks, dips) in the means over time (e.g., from time cluster to time cluster) can be identified, and the changes in the means can indicate particular behavior trends. For instance, a higher mean of CGM values within a time cluster representing a time interval of 9:00 AM to 9:30 AM relative to the mean of CGM values within a time cluster representing a time interval of 8:30 AM to 09:00 AM may indicate a behavior trend of breakfast consumption within the time interval of 9:00 AM to 9:30 AM. As another non-limiting example, a mean of meal insulin values during the 08:00 AM to 8:30 AM interval that exhibits a distinct peak relative to other time-neighboring means of meal insulin values may indicate a behavior trend for the user to administer meal insulin (i.e., a bolus dose) during that specific half-hour window. Accordingly, identifying behavior trends may include identifying at least one of a peak or a dip indicated by the determined mean values of the at least one therapy variable across the plurality of time clusters. As yet another non-limiting example, CGM values within a time cluster representing a time interval of 2:00 PM to 2:30 PM that are significantly lower than the CGM values in neighboring time clusters (e.g., time clusters representing time intervals of 1:30 PM to 02:00 PM and 02:30 PM to 03:00 PM) may indicate a behavior trend of regular exercise during that particular time cluster of the day. In view of the foregoing, identifying at least one behavior trend may include, based at least partially on the identified at least one of a peak or a dip, identifying at least one of a meal event, an exercise event, or a bolus dose.

As additional non-limiting examples, based at least partially on peaks and dips represented in the mean data determined in act 418 of FIG. 4, consistent meal intake times, exercise times, and bolus dosing times may be identified. Furthermore, slopes (e.g., rates of change) of the spikes and dips may indicate exercise rigors, bolus aggressiveness, and/or consistency of meal intake times, exercise times, and bolus dosing times. As non-limiting examples, steeper slopes may indicate one or more of more consistent timing of events (e.g., within the mean data), aggressiveness of bolus doses (e.g., within the therapy data and/or the mean data), and rigor of exercise (e.g., within the therapy data and/or the mean data).

As noted above in regard to FIG. 3, the behavior trends may be associated with particular times of day and/or particular times of day of specific days of a week. Accordingly, the behavior trends may indicate typical meal times, exercise schedules, changes in behavior on weekends relative to weekdays, periodic hormonal variations such as menstrual cycles, recurring events such as job-related periodic travels, and/or psychological stress that tends to occur periodically.

The method 400 may further include, based at least partially on the identified behavior trends, predicting at least one event, as shown in act 406 of FIG. 4. For example, the controller 202 may predict the at least one event. In some embodiments, predicting the at least one event may include predicting at least one of a meal event, an exercise event, or a bolus dose. Furthermore, predicting the at least one event may include predicting at least one of a meal size, an exercise rigor, or a bolus dosing aggressiveness. Moreover, predicting the at least one event may include predicting at least one of a predicted time of day or a predicted time of day and day of the week for the predicted event to occur.

As non-limiting examples, if the identified behavior trends indicate that an event has historically occurred within a given range of time on a given day of the week and/or daily, act 406 may include predicting that the event will occur during a same range of time on a same day of the week and/or daily. Furthermore, depending on the type of event, a probability of the event occurring, may be associated with the predicted event. In some embodiments, the method 400 may only predict events for which the behavior trends indicate that the event has historically occurred above a threshold incidence rate. For instance, the method 400 may include only predict events having a historical incidence rate of occurring over 50% of the time. In additional embodiments, the method 400 may include only predicting events having a historical incidence rate of occurring over 60%, 70%, 80%, or 90% of the time.

In alternative embodiments, the method 400 may include predicting at least substantially all of events indicated by the behavior trends; however, the method 400 may include associating a determined incidence rate with the predicted events.

Responsive to predicting the at least one event, the method 400 may optionally include generating and providing a recommendation to the user to adjust one or more parameters utilized in administering insulin to the user, as shown in act 408 of FIG. 4. For example, the controller 202 of the medicament delivery system 200 may generate a recommendation to adjust the one or more parameters and cause the recommendation to be provided to the user. In some embodiments, the controller 202 may cause the recommendation to be displayed on the user interface 140 of the handheld electronic computing device 106. In additional embodiments, the controller 202 may cause the recommendation to be displayed on a user interface of the automated medicament delivery device 104 and/or the medicament delivery system 200.

The one or more parameters may include amount of bolus doses, recommended bolus dose timings, carb count of meals, basal rates, insulin-to-carbohydrate ratios, insulin sensitivity/correction factors, active insulin times, target blood glucose levels, and total daily insulin doses.

As a non-limiting example, responsive to predicting an event that will increase blood glucose levels (e.g., a meal intake) within a next relatively short period of time (e.g., 3 hrs, 2 hrs, 1 hr, 30 minutes), the controller 202 may generate a recommendation to adjust a basal rate and/or timing or amount of a bolus dose in anticipation of the event. For instance, the controller 202 may generate a recommendation to adjust a basal rate and/or a bolus dose to enable early deliver of insulin in anticipation of the event and better Time in Range (TIR) control. Likewise, responsive to predicting an event that will decrease blood glucose levels (e.g., exercise) within a next relatively short period of time (e.g., 3 hrs, 2 hrs, 1 hr, 30 minutes), the controller 202 may generate a recommendation to adjust a basal rate and/or timing or amount of a bolus dose and adopt a more conservative insulin delivery regime prior to the event. A conservative approach may include lower initial doses, making incremental adjustments, and avoiding aggressive corrections in a time leading up to the forthcoming event. For instance, the controller 202 may generate a recommendation to adjust a basal rate and/or a bolus dose to reduce an amount insulin delivered prior to the event and reduce a risk of exercise-related hypoglycemia.

Referring still to act 408, in some embodiments, the method 400 may require user input to change the parameters. For example, the user made have to manually change the parameters within the memory 128 of the automated medicament delivery device 104 (e.g., within settings of the automated medicament delivery device 104), or the user may have to manually change the parameters in another application on, for example, the handheld electronic computing device 106 or on another device. In additional embodiments, the controller 202 of the medicament delivery system 200 may change the parameters with the memory 128 of the automated medicament delivery device 104 responsive to an user input indicating acceptance of the recommendation by the user.

In some embodiments, responsive to predicting the at least one event, the method 400 may optionally include automatically adjusting parameters utilized in administering insulin to the user, without user input, as shown in act 410 of FIG. 4. For example, the controller 202 of the medicament delivery system 200 may automatically adjust parameters utilized in administering insulin to the user. In other words, the controller 202 may automatically adjust operation of the medicament delivery system 200.

As a non-limiting example, responsive to predicting an event that will increase blood glucose levels (e.g., a meal intake) within a next relatively short period of time (e.g., 3 hrs, 2 hrs, 1 hr, 30 minutes), the controller 202 may automatically adjust a basal rate and/or timing or amount of a bolus dose in anticipation of the event. For instance, the controller 202 may automatically adjust a basal rate and/or a bolus dose to enable early deliver of insulin in anticipation of the event and better Time in Range (TIR) control. Likewise, responsive to predicting an event that will decrease blood glucose levels (e.g., exercise) within a next relatively short period of time (e.g., 3 hrs, 2 hrs, 1 hr, 30 minutes), the controller 202 may automatically adjust a basal rate and/or timing or amount of a bolus dose and adopt a more conservative insulin delivery regime prior to the event. For instance, the controller 202 may automatically adjust a basal rate and/or a bolus dose to reduce an amount insulin delivered prior to the event and reduce a risk of exercise-related hypoglycemia.

In some embodiments, automatically adjusting parameters utilized in administering insulin to the user may include generating at least one request 206 to adjust administration of insulin to the user (e.g., adjust operation of the automated medicament delivery device 104) according to the adjusted parameters, as shown in act 420. Furthermore, in some embodiments, generating the at least one request 206 and act 420 may occur responsive to a user approving the recommendation to adjust one or more parameters described above in regard act 408 of FIG. 4. In one or more embodiments, the controller 202 may generate the at least one request 206. In some embodiments, the request 206 may include instructions to administer basal dosing and/or a bolus dose based on the adjusted parameters.

Additionally, automatically adjusting parameters utilized in administering insulin to the user may include providing the generated at least one request to the automated medicament delivery device 104, as shown in act 422 of FIG. 4. For instance, the controller 202 may provide the at least one request 206 to the delivery mechanism controller 208 of the delivery system 204 of the medicament delivery system 200 via any of the manners described above in regard to FIG. 1 and FIG. 2.

Moreover, the method 400 may optionally include causing the medicament delivery system 200 to deliver insulin to the user's body in accordance with the request 206, as shown in act 424 of FIG. 4. For instance, the method 400 may include causing the medicament delivery system 200 to deliver insulin to the user's body responsive to the request 206 via any of the manners described above in regard to FIG. 1 and FIG. 2. Put another way, the method 400 may include adjusting operation of one or more of the medicament delivery system 200 and/or automated medicament delivery device 104 and causing the medicament delivery system 200 and/or automated medicament delivery device 104 to deliver insulin according to the adjusted operation.

Furthermore, the method 400 may optionally include iteratively repeating act 402 through act 424, as shown in act 426 of FIG. 4. For example, the controller 202 may iteratively repeat act 402 through act 424. In some embodiments, act 402 through act 424 may be repeated every 24 hours. In other embodiments, the act 402 through act 424 may be repeated every 12 hours, 6 hours, 3 hours, 1 hour, 30 minutes, or any other period of time.

Repeating act 402 through act 424 may enable the medicament delivery system 200 to identify new behavior trends and/or changing behavior trends (e.g., a new exercise regime or a new diet). As a result, the medicament delivery system 200 may consistently be analyzing a previous seven-day period using a moving window approach.

### Historical Time Series Prediction Model Features:

One or more embodiments include considering the actual, observed events of a current day, comparing those events to the same times during a previous period of time (e.g., a week), and predicting events based at last partially on the comparison. In particular, some embodiments include considering measurements (e.g., data points) captured on a current day, comparing the measurements to the same times over a past period of time (e.g., a past week), and predicting events based at last partially on the comparison. In particular, using the historical data, events may be predicted for a given prediction horizon (e.g., a next 1 hour to 12 hours) subsequent to the historical data. Moreover, any predictions may be compared to actual, observed events of the current day. By comparing the predictions to what actually occurred, accuracy of the predictions can be determined. Moreover, the accuracy measurements (e.g., errors) may be average to determine how accurate the predictions have been over a past period of time (e.g., a past week) for different time intervals.

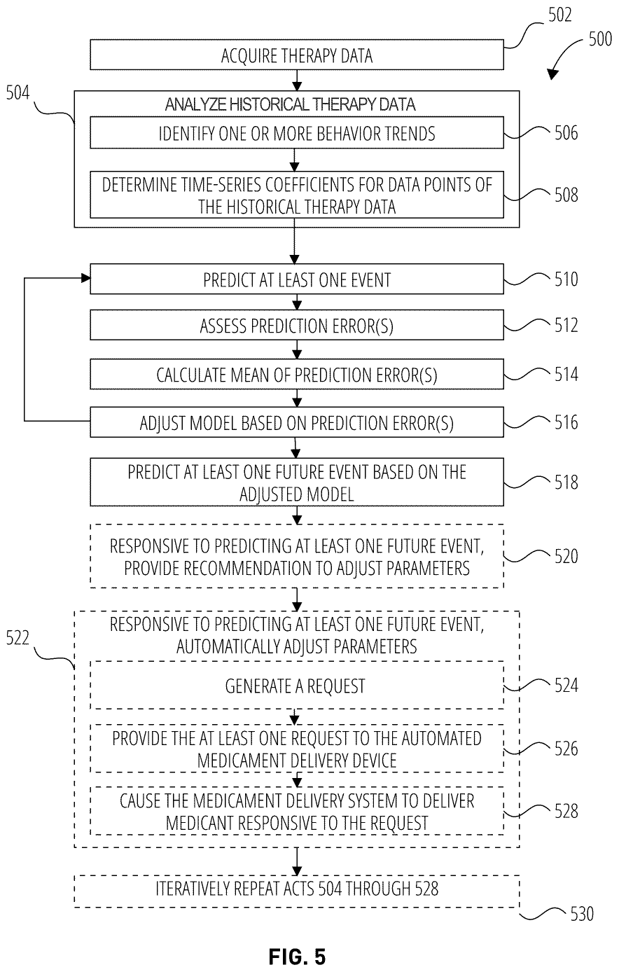

FIG. 5 shows a flowchart of a method 500 of managing medicament therapy for one or more users. In some embodiments, one or more acts of the method 500 may be performed and/or executed by the controller 202 of the medicament delivery system 200. However, the disclosure is not so limited, and one or more acts of the method 500 may be performed, executed, and/or initiated by the delivery mechanism controller 208 of the delivery system 204, other controllers of the system 100, the handheld electronic computing device 106, and/or one or more other remote devices. For purposes of the present disclosure, the acts of the method 500 are described as being performed, executed, and/or initiated by the controller 202 of the medicament delivery system 200.

The method 500 may include acquiring and collecting therapy data associated with a given user, as shown in act 502 of FIG. 5. For example, the controller 202 of the medicament delivery system 200 may acquire the therapy data associated with the given user. In some embodiments, acquiring the therapy data may include acquiring therapy data for a most recent twenty-four-hour (24-hr) period (a “current day”) and at least a previous seven-day period prior to the current day. In additional embodiments, acquiring the therapy data may include acquiring the therapy data for the current day and at least a previous fourteen-day period, a thirty-day period, a sixty-day period, or a longer time period. For the embodiments described herein, a seven-day period is utilized for ease of description; however, it is understood that any time period may be utilized. In view of the foregoing, for purposes of the description of the method 500 and FIG. 5, the therapy data may be separated into 1) historical therapy data including the therapy data representing a period of time (e.g., a seven-day period) prior to the current day (referred to herein as “historical therapy data”) and 2) current day therapy data referred to herein as “current therapy data.”

In some embodiments, the time period for which the therapy data spans may be a moving time period. For instance, the time period may include the current day and a most-recent, seven-day period. In some embodiments, the time period moves (e.g., is updated) every twenty-four hours. In additional embodiments, the time period is constantly changing in real-time and includes a most-recent 192-hour period.

In one or more embodiments, the therapy data may include any of the types of data (e.g., CGM data and/or IOB data) described above in regard to FIG. 3 and FIG. 4.

Responsive to collecting the therapy data, the method 500 may include analyzing the historical therapy data, as shown in act 504 of FIG. 5. For example, the controller 202 may analyze the historical therapy data.

Analyzing the historical therapy data may include identifying one or more behavior trends represented in the historical therapy data, as shown in act 506 of FIG. 5. For example, the controller 202 may identify one or more behavior trends of the user represented in the historical therapy data. The behavior trends may be identified via any of the manners described above in regard to FIG. 3 and FIG. 4.

Furthermore, analyzing the historical therapy data may include determining time-series coefficients for the data points of the historical therapy data (e.g., therapy data representing a previous seven-day (7-day) period), as shown in act 508 of FIG. 5. In one or more embodiments, the controller 202 may determine the time-series coefficients. In some embodiments, the time-series coefficients are estimated. The time-series coefficients may include numerical values that quantify a relationship between values (e.g., data points) within the historical therapy data and correlating values of the current therapy data (e.g., future values). In one or more embodiments, determining time-series coefficients for the data points of the historical therapy data may include determining time series coefficients for portions of the historical therapy data (e.g., data points) associated with identified behavior trends.

In one or more embodiments, the time-series coefficients may be determined via any known method. For example, the time-series coefficients may be determined by fitting a time-series model to the therapy data. In particular, parameters of the time-series model may be estimated, and then the time-series model may be fitted to the therapy data using the estimated parameters. As non-limiting example, the time-series model may include one or more of an autoregressive (AR) model, a moving average (MA) model, an ARIMA model, an exponential smoothing model, or a machine learning model (e.g., an LSTM model, a Prophet model).

The method 500 may further include, based at least partially on the identified behavior trends and utilizing the fitted time-series model and determined time-series coefficients, predicting at least one event, as shown in act 510 of FIG. 5. For example, the controller 202 may predict the at least one event. In some embodiments, predicting at least one event may include utilizing the time-series coefficients to predict at least one event through a prediction horizon. The prediction horizon may include between one hour (1 hr) and twelve hours (12 hrs) beyond (e.g., extending from) the period of time represented in the historical therapy data. For example, the prediction horizon may include at least a portion of the current day. Put yet another way, predicting at least one event may include predicting at least one event for the current day.

Predicting at least one event may include predicting any of the events described above in regard to FIG. 3 and FIG. 4.

Additionally, the method 500 may include determining a prediction error for the predicted at least one event, as shown in act 512 of FIG. 5. For example, the controller 202 may assess a prediction error for the predicted at least one event. In particular, assessing the prediction error may include comparing the predicted at least one event to the current therapy data (i.e., actual observed events) to determine a prediction error.

As a non-limiting example, for predicted CGM values, assessing the prediction error may include utilizing the following Equation 2:

$${CGM}_{actual} = {CGM}_{predicted} \pm error \qquad Equation 2$$

In Equation 2, because CGM_{actual} and CGM_{predicted} are known, the error can be determined.

In some embodiments, assessing a prediction error for the predicted at least one event may include assessing a prediction error for each historical day (e.g., what is predicted based on the historical day) within the prediction horizon. Accordingly, the following metrics may be derived: Error_day1_5 min, Error_day2_5 min, and so on, through the prediction horizon. The foregoing metrics capture a historical accuracy of predictions at specific times of a day.

Moreover, the method 500 may include calculating a mean of the prediction errors for each prediction horizon, as shown in act 514 of FIG. 5. For example, the controller 202 may calculate the mean of the prediction errors for each prediction horizon. For instance, the mean of the prediction errors for each prediction horizon (e.g., 1 hr, 2 hrs, 3 hrs) may be calculated. Calculating the mean of the prediction errors for each prediction horizon results in calculated average errors at given time intervals of the prediction horizons (e.g., 5 minutes, 10 minutes, etc.). The foregoing determined means of the predictions errors represent an average prediction error observed over the past seven days for a given prediction horizon at an exact timestamp (e.g., hour and minute) of the current day.

Based at least partially on the determined prediction error and/or calculated mean of prediction errors, the method 500 may include adjusting the time-series model utilized to predict the at least one event, as shown in act 516 of FIG. 5. For example, the controller 202 may adjust the time-series model. As a non-limiting example, adjusting the time-series model may include adjusting model parameters (e.g., adjusting the time-series coefficients). In one or more embodiments, the parameters of the time-series model may be adjusted via one or more model optimization techniques, such as, for example, Stochastic Gradient Descent, Mini-Batch Gradient Descent, Momentum, Nesterov Accelerated Gradient (NAG), Adagrad (Adaptive Gradient Algorithm), RMSprop (Root Mean Square Propagation), Adam (Adaptive Moment Estimation), Newton's Method, Conjugate Gradient Method, or Bayesian Optimization.

Furthermore, the method 500 may include iteratively repeating act 510 through act 516 to optimize the time-series model utilized to predict events based on the historical therapy data. Put another way, the method 500 may include continuously comparing predicted events with actual events and adjusting the time-series coefficients to improve accuracy of the time-series model over time. Moreover, referring to act 508 through act 516 of FIG. 5 together, the time-series coefficients enable transforming the historical therapy data into a mathematical formula that can predict future events based on the identified behavior trends.

Responsive to adjusting the time-series model, the method 300 may include predicting at least one future event utilizing the adjusted time-series model, as shown in act 518 of FIG. 518. For example, the controller 202 may predicted the at least one future event. The at least on future event may be predicted via any of the manners described above in regard to act 510. Moreover, the at least one future event may include an event beyond the time period of the current therapy data. In other words, the at least one future event may be predicted at a time that has not occurred yet. The at least one future event may include any of the events described above in regard to act 510, FIG. 3, and/or FIG. 4.

Responsive to predicting the at least one future event, the method 500 may optionally include generating and providing a recommendation to the user to adjust one or more parameters utilized in administering insulin to the user, as shown in act 520 of FIG. 5. For example, the controller 202 of the medicament delivery system 200 may generate a recommendation to adjust the one or more parameters and cause the recommendation to be provided to the user. In some embodiments, the controller 202 may cause the recommendation to be displayed on the user interface 140 of the handheld electronic computing device 106. In additional embodiments, the controller 202 may cause the recommendation to be displayed on a user interface of the automated medicament delivery device 104 and/or the medicament delivery system 200.

The one or more parameters may include amount of bolus doses, recommended bolus dose timings, carb count of meals, basal rates, insulin-to-carbohydrate ratios, insulin sensitivity/correction factors, active insulin times, target blood glucose levels, and total daily insulin doses.

As a non-limiting example, responsive to predicting a future event that will increase blood glucose levels (e.g., a meal intake) within a next relatively short period of time (e.g., 3 hrs, 2 hrs, 1 hr, 30 minutes), the controller 202 may generate a recommendation to adjust a basal rate and/or timing or amount of a bolus dose in anticipation of the future event. For instance, the controller 202 may generate a recommendation to adjust a basal rate and/or a bolus dose to enable early deliver of insulin in anticipation of the future event and better Time in Range (TIR) control. Likewise, responsive to predicting a future event that will decrease blood glucose levels (e.g., exercise) within a next relatively short period of time (e.g., 3 hrs, 2 hrs, 1 hr, 30 minutes), the controller 202 may generate a recommendation to adjust a basal rate and/or timing or amount of a bolus dose and adopt a more conservative insulin delivery regime prior to the future event. A conservative approach may include lower initial doses, making incremental adjustments, and avoiding aggressive corrections in a time leading up to the forthcoming future event. For instance, the controller 202 may generate a recommendation to adjust a basal rate and/or a bolus dose to reduce an amount insulin delivered prior to the future event and reduce a risk of exercise-related hypoglycemia.

Referring still to act 520, in some embodiments, the method 500 may require user input to change the parameters. For example, the user made have to manually change the parameters within the memory 128 of the automated medicament delivery device 104 (e.g., within settings of the automated medicament delivery device 104), or the user may have to manually change the parameters in another application on, for example, the handheld electronic computing device 106 or on another device. In additional embodiments, the controller 202 of the medicament delivery system 200 may change the parameters with the memory 128 of the automated medicament delivery device 104 responsive to an user input indicating acceptance of the recommendation by the user.

In some embodiments, responsive to predicting the at least one future event, the method 500 may optionally include automatically adjusting parameters utilized in administering insulin to the user, without user input, as shown in act 522 of FIG. 5. For example, the controller 202 of the medicament delivery system 200 may automatically adjust parameters utilized in administering insulin to the user. In other words, the controller 202 may automatically adjust operation of the medicament delivery system 200.

As a non-limiting example, responsive to predicting a future event that will increase blood glucose levels (e.g., a meal intake) within a next relatively short period of time (e.g., 3 hrs, 2 hrs, 1 hr, 30 minutes), the controller 202 may automatically adjust a basal rate and/or timing or amount of a bolus dose in anticipation of the future event. For instance, the controller 202 may automatically adjust a basal rate and/or a bolus dose to enable early deliver of insulin in anticipation of the future event and better Time in Range (TIR) control. Likewise, responsive to predicting an future event that will decrease blood glucose levels (e.g., exercise) within a next relatively short period of time (e.g., 3 hrs, 2 hrs, 1 hr, 30 minutes), the controller 202 may automatically adjust a basal rate and/or timing or amount of a bolus dose and adopt a more conservative insulin delivery regime prior to the future event. For instance, the controller 202 may automatically adjust a basal rate and/or a bolus dose to reduce an amount insulin delivered prior to the future event and reduce a risk of exercise-related hypoglycemia.

In some embodiments, automatically adjusting parameters utilized in administering insulin to the user may include generating at least one request 206 to adjust administration of insulin to the user (e.g., adjust operation of the automated medicament delivery device 104) according to the adjusted parameters, as shown in act 524. Furthermore, in some embodiments, generating the at least one request 206 and act 524 may occur responsive to a user approving the recommendation to adjust one or more parameters described above in regard act 520 of FIG. 5. In one or more embodiments, the controller 202 may generate the at least one request 206. In some embodiments, the request 206 may include instructions to administer basal dosing and/or a bolus dose based on the adjusted parameters.

Additionally, automatically adjusting parameters utilized in administering insulin to the user may include providing the generated at least one request to the automated medicament delivery device 104, as shown in act 526 of FIG. 5. For instance, the controller 202 may provide the at least one request 206 to the delivery mechanism controller 208 of the delivery system 204 of the medicament delivery system 200 via any of the manners described above in regard to FIG. 1 and FIG. 2.

Moreover, the method 500 may optionally include causing the medicament delivery system 200 to deliver insulin to the user's body in accordance with the request 206, as shown in act 528 of FIG. 5. For instance, the method 500 may include causing the medicament delivery system 200 to deliver insulin to the user's body responsive to the request 206 via any of the manners described above in regard to FIG. 1 and FIG. 2. Put another way, the method 400 may include adjusting operation of one or more of the medicament delivery system 200 and/or automated medicament delivery device 104 and causing the medicament delivery system 200 and/or automated medicament delivery device 104 to deliver insulin according to the adjusted operation.

Furthermore, the method 500 may optionally include iteratively repeating act 502 through act 528, as shown in act 530 of FIG. 5. For example, the controller 202 may iteratively repeat act 502 through act 528. In some embodiments, act 502 through act 528 may be repeated every 24 hours. In other embodiments, the act 502 through act 528 may be repeated every 12 hours, 6 hours, 3 hours, 1 hour, 30 minutes, or any other period of time.

Repeating act 502 through act 528 may enable the medicament delivery system 200 to identify new behavior trends and/or changing behavior trends (e.g., a new exercise regime or a new diet). As a result, the medicament delivery system 200 may consistently be analyzing a previous seven-day period using a moving window approach.

Referring to act 502 through act 530 together, using the method 500 described in regard to FIG. 5 may provide advantages over convention methods of predicting events for insulin therapy. For example, conventional systems utilize prediction models that are static and lack personalization and fail to account for historical user patterns. In contrast, the method 500 described herein develops a time-series model that is personalized and accounts for historical user patterns.

### Pattern Match:

Referring still to method 500, when using historical time series prediction models, it may be assumed that users exhibit repetitive habits with consistent timing. However, this assumption may not always hold true. To address this variability, a pattern-matching method may be employed and added to the method 500, as opposed to relying solely on temporal alignment. The pattern-matching method may improve predictability of events based on current data points (e.g., current measurements) in view of historical data. Accordingly, some embodiments of the present disclosure include adding a pattern-matching method when performing the method 500 described above in regard to FIG. 5.

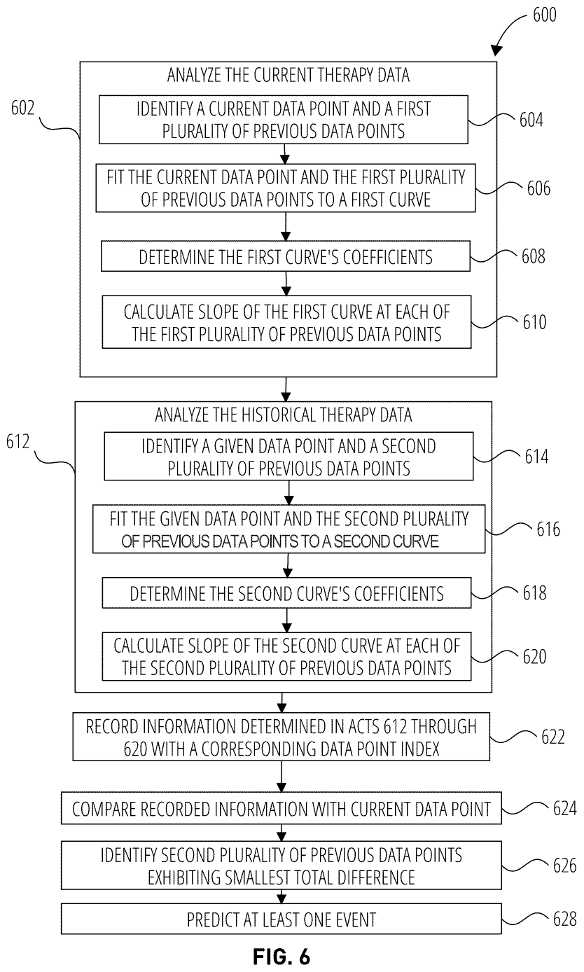

FIG. 6 shows a flowchart of a method 600 of matching patterns depicted in historical therapy data and current therapy data. In some embodiments, the method 600 can be performed subsequent to act 502 or act 508 of FIG. 5; however, the disclosure is not so limited, and the method 600 can be performed at any point between act 502 and act 508 or any other point within method 500.

In some embodiments, one or more acts of the method 600 may be performed and/or executed by the controller 202 of the medicament delivery system 200. However, the disclosure is not so limited, and one or more acts of the method 600 may be performed, executed, and/or initiated by the delivery mechanism controller 208 of the delivery system 204, other controllers of the system 100, the handheld electronic computing device 106, and/or one or more other remote devices. For purposes of the present disclosure, the acts of the method 600 are described as being performed, executed, and/or initiated by the controller 202 of the medicament delivery system 200.

The method 600 may include analyzing the current therapy data, as shown in act 602 of FIG. 6. For example, the controller 202 may analyze the current therapy data. In some embodiments, analyzing the current therapy data may include identifying a current data point and a first plurality of previous data points of the current therapy data immediately preceding the current data point, as shown in act 604 of FIG. 6. As used herein, a “current data point” may include a data point reflecting a most recent measurement or calculation of a given parameter (e.g., CGM value or IOB value). In some embodiments, the first plurality of previous data points includes at least six previous data points. In additional embodiments, the first plurality of previous data points includes at least eight previous data points, twelve previous data points, twenty-four previous data points, or any other number of previous data points.

Analyzing the current therapy data may further include fitting the current data point and the first plurality of previous data points to a first curve, as shown in act 606 of FIG. 6. For example, the controller 202 may fit the current data point and the first plurality of previous data points to a first curve. In some embodiments, fitting the current data point and the first plurality of previous data points to a first curve include calculating a second-order polynomial fitted to the current data point and the first plurality of previous data points. In some embodiments, the second-order polynomial may be represented by ax²+bx+c.

Additionally, analyzing the current therapy data may further include determining the first curve's coefficients, as shown in act 608 of FIG. 6. For example, the controller 202 may determine the first curve's coefficients. In some embodiments, determining the first curve's coefficients may include determining a, b, and c of the second-order polynomial described above in regard to act 606 of FIG. 6. The coefficients may be determined through any known methods.

Moreover, analyzing the current therapy data may further include calculating the slope of the first curve at each of the first plurality of previous data points, as shown in act 610 of FIG. 6. For example, the controller 202 may calculate the slope of the first curve at each of the first plurality of previous data points. In some embodiments, calculating the slope of the first curve at each of the first plurality of previous data points may include calculating the derivative (2ax+b) at each of the first plurality of previous data points.

The method 600 may further include analyzing the historical therapy data, as shown in act 612 of FIG. 6. For example, the controller 202 may analyze the historical therapy data. In some embodiments, analyzing the historical therapy data may include starting at a data point representing seven days (e.g., 168 hrs) prior to the current data point and repeating act 614 through act 620 for each consecutive data point of the historical data. In other words, for historical therapy data representing a seven-day period, acts 614 through 620 may be repeated for all 2016 data points (i.e., 288 data points of each 24-hr period).

Analyzing the historical therapy data may include identifying a given data point and a second plurality of previous data points of the historical therapy data immediately preceding the given data point, as shown in act 614 of FIG. 6. As used herein, a “given data point” may include any data point of the historical therapy data. As noted above, act 614 may first be performed for a data point of the historical therapy data representing seven days (e.g., 168 hrs) prior to the current data point. In some embodiments, identifying the given data point and the second plurality of previous data points may include identifying a given data point and a second plurality of previous data points that reflect measurements and/or calculations of a same parameter reflected by the current data point described above in regard to act 602 of FIG. 6. As a non-limiting example, the given data and the second plurality of previous data points may reflect measurements and/or calculations of CGM values. In some embodiments, the second plurality of previous data points includes at least six previous data points. In additional embodiments, the second plurality of previous data points includes at least eight previous data points, twelve previous data points, twenty-four previous data points, or any other number of previous data points.

Analyzing the historical therapy data may further include fitting the given data point and the second plurality of previous data points to a second curve, as shown in act 616 of FIG. 6. For example, the controller 202 may fit the given data point and the second plurality of previous data points to a second curve. In some embodiments, fitting the given data point and the second plurality of previous data points to a second curve include calculating a second-order polynomial fitted to the given data point and the second plurality of previous data points. In some embodiments, the second-order polynomial may be represented by ax²+bx+c.

Additionally, analyzing the historical therapy data may further include determining the second curve's coefficients, as shown in act 618 of FIG. 6. For example, the controller 202 may determine the second curve's coefficients. In some embodiments, determining the second curve's coefficients may include determining a, b, and c of the second-order polynomial described above in regard to act 616 of FIG. 6. The coefficients may be determined through any known methods.

Moreover, analyzing the historical therapy data may further include calculating the slope of the second curve at each of the second plurality of previous data points, as shown in act 620 of FIG. 6. For example, the controller 202 may calculate the slope of the second curve at each of the second plurality of previous data points. In some embodiments, calculating the slope of the second curve at each of the second plurality of previous data points may include calculating the derivative (2ax+b) at each of the second plurality of previous data points.

The method 600 may further include, in act 622, recording information determined in act 612 through 620 with a corresponding historical data point index. For example, the controller 202 may record the information. In some embodiments, recording the information may include recording the information in a database table and generating the corresponding historical data point index to enable relatively fast retrieval operations.

Referring still to FIG. 6, the method 600 may include comparing the recorded information with the current data point, as shown in act 624. For example, the controller 202 may compare the recorded information with the current data point. For instance, comparing the recorded information with the current data point may include comparing the determined slopes of the first plurality of previous data points with each of the determined slopes of the data points of the historical therapy data. In some embodiments, comparing the determined slopes of the first plurality of previous data points with each of the determined slopes of the data points of the historical therapy data may include comparing the determined slopes of the first plurality of previous data points with each of the determined slopes of each group of consecutive data points analyzed in acts 614 through 620 (i.e., each second plurality of previous data points) of the historical therapy data.

In some embodiments, comparing the determined slopes of the first plurality of previous data points with each of the determined slopes of each group of consecutive data points analyzed in acts 614 through 620 of the historical therapy data may include calculating differences between the slopes of the first plurality of previous data points of the current therapy data and each second plurality of previous data points of the historical therapy data. For example, the differences may be calculated via the following formulas:

$$Diff 1 = abs ( Derivative\_current \_data \_point \_ 1 - Derivative\_historical \_data \_point \_ 1 )$$

$$Diff 2 = abs ( Derivative\_current \_data \_point \_ 2 - Derivative\_historical \_data \_point \_ 2 )$$

$$Diff 3 = abs ( Derivative\_current \_data \_point \_ 3 - Derivative\_historical \_data \_point \_ 3 )$$

$$Diff 4 = abs ( Derivative\_current \_data \_point \_ 4 - Derivative\_historical \_data \_point \_ 4 )$$

$$Diff 5 = abs ( Derivative\_current \_data \_point \_ 5 - Derivative\_historical \_data \_point \_ 5 )$$

$$Diff 6 = abs ( Derivative\_current \_data \_point \_ 6 - Derivative\_historical \_data \_point \_ 6 )$$

where “abs” stands for an absolute value function.

Responsive to determining the differences between the slopes of the first plurality of previous data points of the current therapy data and each second plurality of previous data points of the historical therapy data, the method 600 may include identifying a second plurality of previous data points of the historical therapy data exhibiting a smallest total difference relative to the first plurality of previous data points, as shown in act 626 of FIG. 6. In some embodiments, the controller 202 may identify a second plurality of previous data points of the historical therapy data exhibiting a smallest total difference relative to the first plurality of previous data points. For example, a total difference between the first plurality of previous data points and each plurality of previous data points of the historical therapy data may be determined via the following formula:

$$Total\_Diff = Diff 1 + Diff 2 + Diff 3 + Diff 4 + Diff 5 + Diff 6$$

Identifying the smallest total difference (Smallest_Total_Diff) identifies a historical half-hour period in which all data point derivatives most closely resemble those of the most recent half-hour period in the current therapy data. The second plurality of previous data points exhibiting the smallest total difference is referred to herein as the “matched second plurality of previous data points.”

Furthermore, based at least partially on the comparison of the recorded information with the current data, the method 600 may include predicting at least one event, as shown in act 628 of FIG. 6. For example, the controller 202 predicts at least one event. In particular, by identifying the matched second plurality of previous data points of the historical therapy data, future events may be predicted based at least partially on what events occurred in historical data subsequent to the matched second plurality of previous data points. In some embodiments, predicting at least one event may include predicting values of one or more parameters for up to one-hour in the future.

In some embodiments, the method 600 may further include assessing prediction errors via any of the manners described above in regard to acts 512 and 514 of FIG. 5. Additionally, a prediction error may be determined for the matched second plurality of previous data points, and the prediction error of the matched second plurality of previous data points may be combined with the predicted at least one event. Combining the prediction error of the matched second plurality of previous data points with the predicted at least one event enhances the accuracy of predictions.

Additionally, the method 600 may include any of acts 520 through 530 of FIG. 5.

In the detailed description, reference is made to the accompanying drawings, which form a part hereof, and in which are shown, by way of illustration, specific examples of examples in which the present disclosure may be practiced. These examples are described in sufficient detail to enable a person of ordinary skill in the art to practice the present disclosure. However, other examples may be utilized, and structural, material, and process changes may be made without departing from the scope of the disclosure.

The illustrations presented herein are not meant to be actual views of any particular method, system, device, or structure, but are merely idealized representations that are employed to describe the examples of the present disclosure. The drawings presented herein are not necessarily drawn to scale. Similar structures or components in the various drawings may retain the same or similar numbering for the convenience of the reader; however, the similarity in numbering does not mean that the structures or components are necessarily identical in size, composition, configuration, or any other property.

The description may include examples to help enable one of ordinary skill in the art to practice the disclosed examples. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the examples as generally described herein and illustrated in the drawing could be arranged and designed in a wide variety of different configurations. Thus, the description of various examples is not intended to limit the scope of the present disclosure, but is merely representative of various examples. While the various aspects of the examples may be presented in drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

In the Brief Summary and in the Detailed Description, the claims, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and examples described herein.

Those of ordinary skill in the art would understand that information and signals may be represented using any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative methods, logical blocks, modules, and circuits described in connection with the examples of the system 100, and in particular, the automated medicament delivery device 104 and the handheld electronic computing device 106, disclosed herein may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to examples of the present disclosure.

The examples may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

All references cited herein are incorporated herein in their entireties. If there is a conflict between definitions herein and in an incorporated reference, the definition herein shall control.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternate useful combinations of the elements described, will become apparent to those skilled in the art from the description. Such modifications and embodiments also fall within the scope of the appended claims and equivalents.

What is claimed is:

1. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive therapy data comprising data acquired over a previous at least seven-day period;
analyze the therapy data to identify at least one behavior trend represented in the at least seven-day period;
based at least partially on the identified at least one behavior trend, predict at least one event; and
based at least partially on the predicted at least one event, generate a request to adjust operation of the automated medicament delivery device.

2. The system for administration of claim 1, wherein the therapy data comprises insulin-on-board (IOB) data.

3. The system for administration of claim 1, wherein the therapy data comprises continuous glucose monitoring (CGM) data.

4. The system for administration of claim 1, wherein the predicted at least one event comprises at least one of a meal event, an exercise event, or a bolus dose.

5. The system for administration of claim 4, wherein in the predicted at least one event further comprises at least one of a meal size, an exercise rigor, or a bolus dosing aggressiveness.

6. The system for administration of claim 4, wherein in the predicted at least one event further comprises at least one of a predicted time of day or a predicted time of day and day of the week.

7. The system for administration of claim 1, wherein analyzing the therapy data comprises normalizing the therapy data to determine a probability of the identified behavior trend.

8. The system for administration of claim 1, wherein analyzing the therapy data comprises a dividing the received therapy data into distinct datasets and indexing data points of the datasets based on time.

9. The system for administration of claim 8, wherein each distinct dataset includes therapy data representing a respective 24-hour period of time.

10. The system for administration of claim 8, wherein analyzing the therapy data further comprises, for each time of day represented by the data points of each distinct dataset, determining a mean value of the respective data points of the distinct datasets associated with the given time of day.

11. The system for administration of claim 10, wherein analyzing the therapy data further comprises identifying at least one of a peak or a dip indicated by the determined mean values.

12. The system for administration of claim 11, wherein identifying at least one behavior trend comprises, based at least partially on the identified at least one of a peak or a dip, identifying at least one of a meal event, an exercise event, or a bolus dose.

13. The system for administration of claim 12, wherein identifying at least one behavior trend further comprises identifying at least one of a time of day or a specific day of week and a time of day associated with the identified at least one of a meal event, an exercise event, or a bolus dose.

14. The system for administration of claim 12, wherein identifying at least one behavior trend further comprises identifying at least one of a meal size, an exercise rigor, or a bolus dosing aggressiveness.

15. The system for administration of claim 1, wherein analyzing the therapy data comprises determining statistical features comprising at least one of a first (1st) quartile of the therapy data, a second (2nd) quartile of the therapy data, a third (3rd) quartile of the therapy data, a maximum value of the therapy data, a minimum value of the therapy data, a range between the maximum value and the minimum value of the therapy data, a standard deviation of the therapy data, or a differentiation of the therapy data.

16. The system for administration of claim 1, further comprising instructions that, when executed by the at least one processor, cause the automated medicament delivery device to adjust operation of the automated medicament delivery device according to the generated request.

17. The system for administration of claim 16, further comprising instructions that, when executed by the at least one processor, cause the automated medicament delivery device to deliver medicant according to the adjusted operation.

18. A method for managing medicant delivery to a user, the method comprising:
receiving therapy data comprising data acquired over a previous at least seven-day period;
analyzing the therapy data to identify at least one behavior trend represented in the at least seven-day period;
based at least partially on the identified at least one behavior trend, predicting at least one event; and
providing an indication of the predicted event.

19. The method of claim 18, wherein providing an indication of the predicted event comprises outputting the indication on a handheld electronic computing device.

20. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive therapy data comprising at least one of insulin-on-board data or blood glucose data acquired over a previous at least seven-day period;
analyze the therapy data to identify at least one behavior trend represented in the previous at least seven-day period; and
based at least partially on the identified at least one behavior trend, generate a request to adjust operation of the automated medicament delivery device.

21. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive therapy data comprising data acquired over a previous at least seven-day period;
associate each data point of the therapy data with a time cluster of a plurality of time clusters;
for each time cluster of the plurality of time clusters, extract at least one therapy variable;
determine at least one statistical feature of the at least one therapy variable;
based at least partially on the determined at least one statistical feature, identify at least one behavior trend represented in the at least seven-day period;
based at least partially on the identified at least one behavior trend, predict at least one event; and
based at least partially on the predicted at least one event, generate a request to adjust operation of the automated medicament delivery device.

22. The system for administration of claim 21, wherein the therapy data comprises insulin-on-board (IOB) data.

23. The system for administration of claim 21, wherein the therapy data comprises continuous glucose monitoring (CGM) data.

24. The system for administration of claim 21, wherein the predicted at least one event comprises at least one of a meal event, an exercise event, or a bolus dose.

25. The system for administration of claim 24, wherein in the predicted at least one event further comprises at least one of a meal size, an exercise rigor, or a bolus dosing aggressiveness.

26. The system for administration of claim 24, wherein in the predicted at least one event further comprises at least one of a predicted time of day or a predicted time of day and day of the week.

27. The system for administration of claim 21, wherein each time cluster of the plurality of time cluster represents a thirty-minute (30-minute) time interval.

28. The system for administration of claim 21, wherein the plurality of time clusters comprise forty-eight (48) time clusters and represents a twenty-four-hour (24-hr) period.

29. The system for administration of claim 28, wherein extracting at least one therapy variable comprises extracting at least one of CGM values, derivatives of the CGM values, meal insulin values, algorithm-derived insulin values, correction insulin-on-board (IOB) values, TDI (Total Daily Insulin) values, meal IOB values, algorithm IOB values, or total IOB values represented within each time cluster of the plurality of time clusters.

30. The system for administration of claim 28, wherein determining at least one statistical feature of the at least one therapy variable comprises determining a mean value of the respective data points associated with at least one therapy variable of each time cluster of the plurality of time clusters.

31. The system for administration of claim 30, further comprising identifying at least one of a peak or a dip indicated by the determined mean values of the at least one therapy variable across the plurality of time clusters.

32. The system for administration of claim 31, wherein identifying at least one behavior trend comprises, based at least partially on the identified at least one of a peak or a dip, identifying at least one of a meal event, an exercise event, or a bolus dose.

33. The system for administration of claim 32, wherein identifying at least one behavior trend further comprises identifying at least one of a time of day or a specific day of week and a time of day associated with the identified at least one of a meal event, an exercise event, or a bolus dose.

34. The system for administration of claim 32, wherein identifying at least one behavior trend further comprises identifying at least one of a meal size, an exercise rigor, or a bolus dosing aggressiveness.

35. The system for administration of claim 21, wherein determining at least one statistical feature of the at least one therapy variable comprises determining, for the data points associated with the at least one therapy variable, at least one of a first (1st) quartile, a second (2nd) quartile, a third (3rd) quartile, a maximum value, a minimum value, a range between the maximum value and the minimum value, a standard deviation, or a differentiation.

36. The system for administration of claim 21, further comprising instructions that, when executed by the at least one processor, cause the automated medicament delivery device to adjust operation of the automated medicament delivery device according to the generated request.

37. The system for administration of claim 36, further comprising instructions that, when executed by the at least one processor, cause the automated medicament delivery device to deliver medicant according to the adjusted operation.

38. A method for managing medicant delivery to a user, the method comprising:
receiving therapy data comprising data acquired over a previous at least seven-day period;
associating each data point of the therapy data with a time cluster of a plurality of time clusters;
for each time cluster of the plurality of time clusters, extracting at least one therapy variable;
determining at least one statistical feature of the at least one therapy variable;
based at least partially on the determined at least one statistical feature, identifying at least one behavior trend represented in the at least seven-day period;
based at least partially on the identified at least one behavior trend, predicting at least one event; and
providing an indication of the predicted event.

39. The method of claim 38, wherein providing an indication of the predicted event comprises outputting the indication on a handheld electronic computing device.

40. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive therapy data comprising at least one of insulin-on-board data or blood glucose data acquired over a previous at least seven-day period;
associate each data point of the therapy data with a time cluster of a plurality of time clusters;
for each time cluster of the plurality of time clusters, extract at least one therapy variable;
determine at least one statistical feature of the at least one therapy variable;
based at least partially on the determined at least one statistical feature, identify at least one behavior trend represented in the at least seven-day period; and
based at least partially on the identified at least one behavior trend, generate a request to adjust operation of the automated medicament delivery device.

41. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive current therapy data comprising data acquired over a most recent 24-hr period;
receive historical therapy data comprising data acquired over at least a seven-day period prior to the 24-hr period;
estimate time-series coefficients for data points of the historical therapy data by fitting a time-series model to the data points of the historical therapy data;
based at least partially on the determined time-series coefficients, predict at least one event;
based at least partially on the current therapy data, assess a prediction error of the predicted at least one event;
adjust the time-series model based at least partially on the assessed prediction error;
based on the adjusted time-series model, predict at least one future event; and
based at least partially on the predicted at least one future event, generate a request to adjust operation of the automated medicament delivery device.

42. The system for administration of claim 41, wherein the current therapy data and the historical therapy data comprise insulin-on-board (IOB) data.

43. The system for administration of claim 41, wherein the current therapy data and the historical therapy data comprise continuous glucose monitoring (CGM) data.

44. The system for administration of claim 41, wherein the predicted at least one event comprises at least one of a meal event, an exercise event, or a bolus dose.

45. The system for administration of claim 44, wherein in the predicted at least one event further comprises at least one of a meal size, an exercise rigor, or a bolus dosing aggressiveness.

46. The system for administration of claim 44, wherein in the predicted at least one event further comprises at least one of a predicted time of day or a predicted time of day and day of the week.

47. The system for administration of claim 41, wherein the time-series model comprises one of an autoregressive (AR) model, a moving average (MA) model, an ARIMA model, an exponential smoothing model, or a machine learning model.

48. The system for administration of claim 41, further comprising instructions that, when executed by the at least one processor, cause the automated medicament delivery device to:
identify at least one behavior trend from the historical therapy data; and
predict the at least one event based at least partially on the identified at least one behavior trend.

49. The system for administration of claim 48, wherein predicting at least one event comprises predicting parameter values for a prediction horizon of at least one hour.

50. The system for administration of claim 49, wherein the parameter values comprise one or more of CGM values or IOB values.

51. The system for administration of claim 48, wherein predicting at least one event comprises predicting parameter values for a prediction horizon of at least twelve hours.

52. The system for administration of claim 41, further comprising instructions that, when executed by the at least one processor, cause the automated medicament delivery device to identify at least one behavior trend from the historical therapy data.

53. The system for administration of claim 52, wherein identifying at least one behavior trend comprises identifying at least one of a meal event, an exercise event, or a bolus dose.

54. The system for administration of claim 53, wherein identifying at least one behavior trend further comprises identifying at least one of a time of day or a specific day of week and a time of day associated with the identified at least one of a meal event, an exercise event, or a bolus dose.

55. The system for administration of claim 53, wherein identifying at least one behavior trend further comprises identifying at least one of a meal size, an exercise rigor, or a bolus dosing aggressiveness.

56. The system for administration of claim 41, further comprising instructions that, when executed by the at least one processor, cause the automated medicament delivery device to adjust operation of the automated medicament delivery device according to the generated request.

57. The system for administration of claim 56, further comprising instructions that, when executed by the at least one processor, cause the automated medicament delivery device to deliver medicant according to the adjusted operation.

58. A method for managing medicant delivery to a user, the method comprising:
receiving historical therapy data comprising data acquired over at least a seven-day period prior to a current 24-hr period;
fitting a time-series model to the data points of the historical therapy data;
utilizing the fitted time-series model to predict at least one event; and
providing an indication of the predicted event.

59. The method of claim 58, wherein providing an indication of the predicted event comprises outputting the indication on a handheld electronic computing device.

60. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive historical therapy data comprising data acquired over at least a seven-day period;
fit a time-series model to the data points of the historical therapy data;
utilize the fitted time-series model to predict at least one event; and
based at least partially on the predicted at least one event, generate a request to adjust operation of the automated medicament delivery device.

61. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive current therapy data comprising data acquired over a most recent 24-hr period;
receive historical therapy data comprising data acquired over at least a seven-day period prior to the 24-hr period;
fit at least a portion of the current therapy data to a first curve;
fit at least one portion of the historical therapy data to at least one second curve;
compare the first curve to the at least one second curve;
based at least partially on the comparison of the first curve to the at least one second curve, predict at least one event; and
based at least partially on the predicted at least one event, generate a request to adjust operation of the automated medicament delivery device.

62. The system for administration of claim 61, wherein the current therapy data and the historical therapy data comprise insulin-on-board (IOB) data.

63. The system for administration of claim 61, wherein the current therapy data and the historical therapy data comprise continuous glucose monitoring (CGM) data.

64. The system for administration of claim 61, wherein the predicted at least one event comprises at least one of a meal event, an exercise event, or a bolus dose.

65. The system for administration of claim 64, wherein in the predicted at least one event further comprises at least one of a meal size, an exercise rigor, or a bolus dosing aggressiveness.

66. The system for administration of claim 64, wherein in the predicted at least one event further comprises at least one of a predicted time of day or a predicted time of day and day of the week.

67. The system for administration of claim 61, wherein fitting at least a portion of the current therapy data to a first curve comprises:
identifying a current data point and a first plurality of previous data points of the current therapy data;
fitting the current data point and the first plurality of previous data points to the first curve;
determining the first curve's coefficients; and
calculating slopes of the first curve at each of the first plurality of previous data points.

68. The system for administration of claim 67, wherein fitting at least one portion of the historical therapy data to at least one second curve comprises:
identifying a given data point and a second plurality of previous data points of the historical therapy data;
fitting the given data point and the second plurality of previous data points to the at least one second curve;
determining the at least one second curve's coefficients; and
calculating slopes of the at least one second curve at each of the second plurality of previous data points.

69. The system for administration of claim 68, wherein comparing the first curve to the at least one second curve comprises comparing the calculated slopes of the first curve to the calculated slopes of the at least one second curve.

70. The system for administration of claim 68, wherein predicting at least one event comprises predicting parameter values for a prediction horizon of at least one hour.

71. The system for administration of claim 70, wherein the parameter values comprise one or more of CGM values or IOB values.

72. The system for administration of claim 61, wherein fitting at least one portion of the historical therapy data to at least one second curve comprises fitting each of a plurality of portions of the historical therapy data to a respective second curve of a plurality of second curves.

73. The system for administration of claim 72, wherein the plurality of portions of the historical therapy data comprises 2016 different portions historical therapy data.

74. The system for administration of claim 72, further comprising instructions that, when executed by the at least one processor, cause the automated medicament delivery device to identify a second curve of the plurality of second curves exhibiting a smallest total difference relative to the first curve.

75. The system for administration of claim 74, wherein the at least one event is predicted based at least partially on the identified second curve of the plurality of second curves exhibiting a smallest total difference relative to the first curve.

76. The system for administration of claim 74, further comprising instructions that, when executed by the at least one processor, cause the automated medicament delivery device to determine a prediction error of the at least one predicted event based on actual, observed events.

77. The system for administration of claim 61, further comprising instructions that, when executed by the at least one processor, cause the automated medicament delivery device to adjust operation of the automated medicament delivery device according to the generated request.

78. The system for administration of claim 77, further comprising instructions that, when executed by the at least one processor, cause the automated medicament delivery device to deliver medicant according to the adjusted operation.

79. A method for managing medicant delivery to a user, the method comprising:
receiving current therapy data comprising data acquired over a most recent 24-hr period;
receiving historical therapy data comprising data acquired over at least a seven-day period prior to the 24-hr period;
fitting at least a portion of the current therapy data to a first curve;
fitting at least one portion of the historical therapy data to at least one second curve;
comparing the first curve to the at least one second curve;
based at least partially on the comparison of the first curve to the at least one second curve, predicting at least one event; and
providing an indication of the predicted event.

80. The method of claim 79, wherein providing an indication of the predicted event comprises outputting the indication on a handheld electronic computing device.

81. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
receive current therapy data comprising data acquired over a most recent 24-hr period;
receive historical therapy data comprising data acquired over at least a seven-day period prior to the 24-hr period;
fit at least a portion of the current therapy data to a first curve;
fit each of a plurality of portions of the historical therapy data to a respective second curve of a plurality of second curves;
compare the first curve to each of the plurality of second curves;
identify a second curve of the plurality of second curves that exhibit a smallest total difference relative to the first curve;
based at least partially on the identified second curve, predict at least one event; and
based at least partially on the predicted at least one event, generate a request to adjust operation of the automated medicament delivery device.
